(19) United States
(12) Patent Application Publication
(10) Pub. No.: US 2024/0369483 A1
(43) Pub. Date: Nov. 7, 2024

(54) Assessing UV Germicidal Effectiveness Using DNA Tags

(71) Applicant: SafeTraces, Inc., Pleasanton, CA (US)

(72) Inventors: Nicholas Heredia, Mountain Home, CA (US); Phil M. Arnold, Redwood City, CA (US); Ashley Fry, Lathrop, CA (US); Nilson Palma, Pleasanton, CA (US)

(21) Appl. No.: 18/624,357

(22) Filed: Apr. 2, 2024

Related U.S. Application Data
(60) Provisional application No. 63/609,233, filed on Dec. 12, 2023.
(60) Provisional application No. 63/499,629, filed on May 2, 2023.

(51) Int. Cl. G01N 21/64; C12Q 1/6806; C12Q 1/6876

(52) U.S. Cl. CPC G01N 21/6428; C12Q 1/6806; C12Q 1/6876; G01N 2021/6439

(57) ABSTRACT

A germicidal verification system might comprise a dispersing unit for dispersing a tag composition, wherein the tag composition comprises two or more tags, a tag comprising a nonpathogenic portion having a particular range of sensitivity to exposure to ultraviolet (UV) radiation, wherein not all tags of the two or more tags have the same range of sensitivity. A dispersed tag composition, dispersed into an environment, can be collected after being exposed at least in part to some UV radiation in the environment. By measuring amounts of the two or more tags in the collected tag composition, wherein measurements of the measurement unit of a given tag depend on an amount of sensitivity to UV exposure of the given tag, effectiveness of UV radiation as a germicide could be measured. Relative degradation among tags can be measured and used as a proxy for, among other things, pathogens in the environment.

### CROSS-REFERENCE TO RELATED APPLICATION

This application claims the benefit of and priority from, U.S. Provisional Patent Application No. 63/499,629 filed May 2, 2023, entitled “Assessing UV Germicidal Effectiveness Using DNA Tags”, and claims the benefit of and priority from, U.S. Provisional Patent Application No. 63/609,233 filed Dec. 12, 2023, entitled “Assessing UV Germicidal Effectiveness Using DNA Tags”.

### FIELD

The present disclosure generally relates to measuring germicidal effectiveness and more particularly to using DNA tags, aerosolized or otherwise, in measuring or estimating UV germicidal effectiveness.

### BACKGROUND

In response to pandemics and the need for sterile and/or sanitary conditions in enclosed spaces and elsewhere, there is a growing market for equipment that can be used to improve such spaces. A number of technologies are available for air filtration, sanitizing, and processing air and other fluids. One approach uses ultraviolet (UV) light to generate a germicidal effect.

For example, airflow in a building's heating, ventilation, and air conditioning (HVAC) infrastructure might be directed through a UV germicidal irradiation (UVGI) chamber in an HVAC installation that exposes the air to intense UV light that can kill or disable bacteria and viruses that might otherwise be harmful to building occupants. Other UVGI installations might use UV lights that might shine in spaces not occupied by humans or other desired occupants, such as by positioning UV lights towards a ceiling such that the air in a room is exposed but not people walking around on the floor. Where there is a need for a UV germicidal effect on surfaces cohabited by humans, lower levels of UV radiation might be used.

Many variables go into the effectiveness of a UVGI installation and effectiveness might vary from instance to instance and over time. To show effectiveness of particular equipment, a manufacturer might test each apparatus before shipping by exposing the apparatus to a pathogen, applying UV light, and measuring a reduction in live pathogen as a result. While the apparatus can then be thoroughly sanitized, customers might not want to receive an apparatus that has been exposed to actual pathogens.

An alternative approach is to test one instance of a particular model of apparatus (such as a UV airflow chamber, UV light, etc.) with an actual pathogen and infer that all other manufactured instances of that model would have the same behavior. This would have limitations, as manufacturing variabilities might not be taken into account and effectiveness of equipment over time would not be accounted for. In addition, installation details and techniques at a particular location could factor into effectiveness of the equipment.

There is a need to provide measures of effectiveness of UVGI equipment, possibly in situ, and over time, that does not require the use of actual pathogens.

### REFERENCES

[1] N. H. L. Leung, Transmissibility and transmission of respiratory viruses, Nat. Rev. Microbiol. 19 (2021). https://doi.org/10.1038/s41579-021-00535-6.
[2] B. F. Leo, C. Y. Lin, K. Markandan, L. H. Saw, M. S. Mohd Nadzir, K. Govindaraju, I. I. Shariffuddin, R. Sankara, Y. W. Tiong, H. Pakalapati, M. Khalid, An overview of SARS-CoV-2 transmission and engineering strategies to mitigate risk, J. Build. Eng. 73 (2023) 106737. https://doi.org/10.1016/j.jobc.2023.106737.
[3] E. A. Nardell, S. J. Bucher, P. W. Brickner, C. Wang, R. L. Vincent, K. Becan-McBride, M. A. James, M. Michael, J. D. Wright, Safety of upper-room ultraviolet germicidal air disinfection for room occupants: Results from the Tuberculosis Ultraviolet Shelter Study, Public Health Rep. 123 (2008) 52-60. https://doi.org/10.1177/003335490812300108.
[4] F. Ibrahim, E. Z. Samsudin, A. R. Ishak, J. Sathasivam, Hospital indoor air quality and its relationships with building design, building operation, and occupant-related factors: A mini-review, Front. Public Heal. 10 (2022). https://doi.org/10.3389/fpubh.2022.1067764.
[5] C. A. Gilkeson, C. J. Noakes, M. A. I. Khan, Computational fluid dynamics modelling and optimisation of an upper-room ultraviolet germicidal irradiation system in a naturally ventilated hospital ward, Indoor Built Environ. 23 (2014). https://doi.org/https://doi.org/10.1177/1420326X14532933.
[6] A. Alani, I. E. Barton, M. J. Seymour, L. C. Wrobel, Application of Lagrangian particle transport model to tuberculosis (TB) bacteria UV dosing in a ventilated isolation room, Int. J. Environ. Health Res. 11 (2001). https://doi.org/10.1080/09603120020047000.
[7] C. C. Wang, K. A. Prather, J. Sznitman, J. L. Jimenez, S. S. Lakdawala, Z. Tufekci, L. C. Marr, Airborne transmission of respiratory viruses, Science (80-.). 373 (2021). https://doi.org/10.1126/science.abd9149.
[8] G. R. Johnson, L. Morawska, Z. D. Ristovski, M. Hargreaves, K. Mengersen, C. Y. H. Chao, M. P. Wan, Y. Li, X. Xie, D. Katoshevski, S. Corbett, Modality of human expired aerosol size distributions, J. Aerosol Sci. 42 (2011) 839-851. https://doi.org/10.1016/j.jaerosci.2011.7.009.
[9] N. Clements, I. Arvelo, P. Arnold, N.J. Heredia, U. W. Hodges, S. Deresinski, P. W. Cook, K. A. Hamilton, Informing Building Strategies to Reduce Infectious Acrosol Transmission Risk by Integrating DNA Aerosol Tracers with Quantitative Microbial Risk Assessment, Environ. Sci. Technol. 57 (2023) 5771-5781. https://doi.org/10.1021/acs.est.2c08131.
[10] W. Kowalski, Ultraviolet germicidal irradiation handbook: UVGI for air and surface disinfection, 2009. https://doi.org/10.1007/978-3-642-01999-9.
[11] R. N. Harding, C. A. Hara, S. B. Hall, E. A. Vitalis, C. B. Thomas, A. D. Jones, J. A. Day, V. R. Tur-Rojas, T. Jorgensen, E. Herchert, R. Yoder, E. K. Wheeler, G. R. Farquar, Unique DNA-barcoded aerosol test particles for studying aerosol transport, Aerosol Sci. Technol. 50 (2016) 429-435. https://doi.org/10.1080/02786826.2016.1162903.
[12] I. Arvelo, F. Pagone, J. Persky, C. E. Carpio, P. Arnold, N. Clements, Decay rates of two tracer gases compared to DNA-tagged liquid aerosol tracer particles: Impact of varying dilution rate and filtration, Build. Environ. 212 (2022) 108819. https://doi.org/10.1016/j.buildenv.2022.108819.
[13] M. G. Kemp, A. Sancar, DNA excision repair: Where do all the dimers go?, Cell Cycle 11 (2012) 2997-3002. https://doi.org/10.4161/cc.21126.
[14] L. H. F. Mullenders, A.-M. Hazekamp-van Dokkum, W. H. J. Kalle, H. Vrieling, M. Z. Zdzienicka, A. A. van Zeeland, UV-induced photolesions, their repair and mutations, Mutat. Res. Toxicol. 299 (1993) 271-276. https://doi.org/10.1016/0165-1218 (93) 90103-K.
[15] J. A. Sikorsky, D. A. Primerano, T. W. Fenger, J. Denvir, DNA damage reduces Taq DNA polymerase fidelity and PCR amplification efficiency, Biochem. Biophys. Res. Commun. 355 (2007) 431-437. https://doi.org/10.1016/j.bbrc.2007.1.169.
[16] M. Otaki, Y. Higashino, Y. Yamada, Experimental validation of determinants of UV sensitivity using synthetic DNA, J. Photochem. Photobiol. 12 (2022) 100139. https://doi.org/10.1016/j.jpap.2022.100139.
[17] S. Lim, E. R. Blatchley, UV Dose-Response Behavior of Air-Exposed Microorganisms, J. Environ. Eng. 138 (2012) 780-785. https://doi.org/10.1061/(ASCE) EE.1943-7870.0000535.
[18] M. Buonanno, D. Welch, I. Shuryak, D. J. Brenner, Far-UVC light (222 nm) efficiently and safely inactivates airborne human coronaviruses, Sci. Rep. 10 (2020) 1-8. https://doi.org/10.1038/s41598-020-67211-2.
[19] D. Welch, M. Buonanno, V. Grilj, I. Shuryak, C. Crickmore, A. W. Bigelow, G. Randers-Pehrson, G. W. Johnson, D. J. Brenner, Far-UVC light: A new tool to control the spread of airborne-mediated microbial diseases, Sci. Rep. 8 (2018) 1-7. https://doi.org/10.1038/s41598-018-21058-w.
[20] E. R. Blatchley, D. J. Brenner, H. Claus, T. E. Cowan, K. G. Linden, Y. Liu, T. Mao, S. J. Park, P. J. Piper, R. M. Simons, D. H. Sliney, Far UV-C radiation: An emerging tool for pandemic control, Crit. Rev. Environ. Sci. Technol. 53 (2023) 733-753. https://doi.org/10.1080/10643389.2022.2084315.
[21] B. J. Hindson, K. D. Ness, D. A. Masquelier, P. Belgrader, N. J. Heredia, A. J. Makarewicz, I. J. Bright, M. Y. Lucero, A. L. Hiddessen, T. C. Legler, T. K. Kitano, M. R. Hodel, J. F. Petersen, P. W. Wyatt, E. R. Steenblock, P. H. Shah, L. J. Bousse, C. B. Troup, J. C. Mellen, D. K. Wittmann, N. G. Erndt, T. H. Cauley, R. T. Kochler, A. P. So, S. Dube, K. A. Rose, L. Montesclaros, S. Wang, D. P. Stumbo, S. P. Hodges, S. Romine, F. P. Milanovich, H. E. White, J. F. Regan, G. A. Karlin-Neumann, C. M. Hindson, S. Saxonov, B. W. Colston, High-throughput droplet digital PCR system for absolute quantitation of DNA copy number, Anal. Chem. 83 (2011) 8604-8610. https://doi.org/10.1021/ac202028g.
A[22] Mazzocco, T. E. Waddell, E. Lingohr, R. P. Johnson, Enumeration of Bacteriophages Using the Small Drop Plaque Assay System, in: 2009: pp. 81-85. https://doi.org/10.1007/978-1-60327-164-6_9.
[23] R Core Team, R: A Language and Environment for Statistical Computing, (2020). https://www.r-project.org/.
[24] H. Wickham, M. Averick, J. Bryan, W. Chang, L. McGowan, R. François, G. Grolemund, A. Hayes, L. Henry, J. Hester, M. Kuhn, T. Pedersen, E. Miller, S. Bache, K. Müller, J. Ooms, D. Robinson, D. Seidel, V. Spinu, K. Takahashi, D. Vaughan, C. Wilke, K. Woo, H. Yutani, Welcome to the Tidyverse, J. Open Source Softw. 4 (2019) 1686. https://doi.org/10.21105/joss.01686.
[25] E. J. Dietz, W. Mendenhall, T. Sincich, A Second Course in Statistics: Regression Analysis, J. Am. Stat. Assoc. 92 (1997) 797. https://doi.org/10.2307/2965740.
[26] ASHRAE, Standard 241, Control of Infectious Aerosols, (2023).
[27] ASTM International, ASTM E741-11 (2017) Standard Test Method for Determining Air Change in a Single Zone by Means of a Tracer Gas Dilution, ASTM, West Conshohocken, 2017.
[28] The Pennsylvania State University, Applied Regression Analysis. Identifying Influential Data Points, (2018). https://online.stat.psu.edu/stat462/node/173/(accessed Jan. 26, 2024).
[29] G. Chevrefils, E. Caron, H. Wright, G. Sakamoto, UV Dose Required to Achieve Incremental Log Inactivation of Bacteria, Protozoa and Viruses, (2006) 38-45. https://www.uvclight.co.uk/media/dosage_for_uvc.pdf (accessed Feb. 16, 2024).
[30] Y. K. Law, R. A. Forties, X. Liu, M. G. Poirier, B. Kohler, Sequence-dependent thymine dimer formation and photoreversal rates in double-stranded DNA, Photochem. Photobiol. Sci. 12 (2013) 1431-1439. https://doi.org/10.1039/c3U.S. Plant Pat. No. 50,078k.
[31] E. R. Blatchley, Photochemical Reactors: Theory, Methods, and Applications of Ultraviolet Radiation, 1st ed., Wiley, Hoboken, 2023.
[32] Center for Disease Control and Prevention, Ventilation in Buildings, (2023). https://www.cdc.gov/coronavirus/2019-ncov/community/ventilation.html #print] (accessed Feb. 16, 2024).
[33] E. Kujundzic, F. Matalkah, C. J. Howard, M. Hernandez, S. L. Miller, UV Air Cleaners and Upper-Room Air Ultraviolet Germicidal Irradiation for Controlling Airborne Bacteria and Fungal Spores, J. Occup. Environ. Hyg. 3 (2006) 536-546. https://doi.org/10.1080/15459620600909799.

All references, including publications, patent applications, and patents, cited herein are hereby incorporated by reference to the same extent as if each reference were individually and specifically indicated to be incorporated by reference and were set forth in its entirety herein.

### SUMMARY

A germicidal verification system might comprise a dispersing unit for dispersing a tag composition, wherein the tag composition comprises two or more tags, a tag comprising a nonpathogenic portion having a particular range of sensitivity to exposure to ultraviolet (UV) radiation, wherein not all tags of the two or more tags have the same range of sensitivity, a conduit for providing dispersed tags in the tag composition, for being dispersed into an environment, a collection unit for collecting collected tag composition, wherein the collected tag composition comprises portions of the dispersed tags dispersed into the environment, exposed at least in part to some UV radiation in the environment, and then collected by the collection unit, and a measurement unit for measuring amounts of the two or more tags in the collected tag composition, wherein measurements of the measurement unit of a given tag depend, at least in part, on an amount of sensitivity to UV exposure of the given tag.

The sensitivity to UV exposure of a given tag might be due to UV radiation degradation of the given tag. Tags of the two or more tags might comprise noncoding strings of nucleic acid base pairs, wherein the noncoding strings of nucleic acid base pairs are selected from the group of DNA sequences and RNA sequences.

The conduit might be adapted to disperse tags into or upon air, water, and/or a material that is to be sanitized.

The measurements of the measurement unit might measure concentrations of pathogen surrogates having differing UV sensitivities, whereby changes in pathogen surrogate concentrations due to UV exposure can be distinguished from changes in concentrations due to air mixing, dilution, flow obstructions, and/or introduction of non-tag materials.

The tag might comprise a primer portion, a tag identifying sequence, and a dose-responsive sequence, wherein the tag composition comprises at least two tags that have different dose-responsive sequences, and/or wherein the different dose-responsive sequences are base pair sequences selected to control for sensitivity of a tag to UV degradation. The primer portion might be selected to be relatively highly robust to UV exposure.

The tags might be deployed in an aerosol form to be mixed with air that is to be sanitized. The tags might be deployed in a solid form and applied to coupons attachable to surfaces, wherein the coupons are formed from a substrate having tags attached thereto, suitable for application to a surface to be sanitized using UV radiation exposure.

The measurement unit for measuring amounts might measure a first original concentration of a first tag, a second original concentration of a second tag, a first post-exposure concentration of the first tag, and a second post-exposure concentration of the second tag, and determines a UV germicidal effectiveness based on original versus measured concentrations of the first tag compared to original versus measured concentrations of the second tag, wherein the first tag and the second tag have differing robustness to UV exposure.

The tags might be selected to have robustness to UV exposure that is determined based on an amount of expected UV exposure in the environment.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of methods and apparatus, as defined in the claims, is provided in the following written description of various embodiments of the disclosure and illustrated in the accompanying drawings.

### BRIEF DESCRIPTION OF THE DRAWINGS

Various embodiments in accordance with the present disclosure will be described with reference to the drawings, in which:

FIG. 1 is a diagram of a germicidal effectiveness testing configuration, according to various embodiments.

FIG. 2 is a flowchart of a testing process, according to various embodiments.

FIG. 3 illustrates various tags that might be used in a testing process, according to various embodiments.

FIG. 4 is a plot, according to various embodiments.

FIG. 5 is a plot, according to various embodiments.

FIG. 6 is a plot, according to various embodiments.

FIG. 7 is a plot, according to various embodiments.

FIG. 8 is a plot, according to various embodiments.

FIG. 9 is a plot, according to various embodiments.

FIG. 10 shows plots of results of UV exposure experiments on foil coupons.

FIG. 11 shows plots of results of UV exposure effects on DNA tag decay rates on coupons.

FIG. 12 shows plots of UV effects on aerosolized tags as eACH.

FIG. 13 shows plots of UV effects on bacteriophage MS2 as eACH.

FIG. 14 illustrates an example of how using an air cleaner provides a measurable eACH.

FIG. 15 illustrates an example of foil coupons having tracers deposited thereon.

FIG. 16 illustrates an experimental chamber setup.

FIG. 17 illustrates an air sampling timeline.

FIG. 18 provides plots of results of UV exposure experiments on foil coupons, focusing on the UV-sensitive and resistant tags.

FIG. 19 illustrates UV Inactivation curves in logarithmic scale for aerosolized MS2 bacteriophage in a sealed chamber.

FIG. 20 illustrates UV Inactivation curves in logarithmic scale for aerosolized DNA Tag-D1 in a sealed chamber.

FIG. 21 illustrates UV Inactivation curves in logarithmic scale for aerosolized DNA Tag-LM4 in a sealed chamber.

FIG. 22 illustrates UVGI configuration at a commercial conference room for effectiveness evaluation.

FIG. 23 illustrates UV Inactivation curves in logarithmic scale for aerosolized DNA Tag-D1 in a conference room in a commercial building.

FIG. 24 illustrates UV Inactivation curves in logarithmic scale for aerosolized DNA Tag-LM4 in a conference room in a commercial building.

FIG. 25 illustrates scaling factors for MS2 and particular DNA tracer tags.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

Airborne exposure to pathogens, including viruses like coronaviruses (responsible for COVID-19 and some common cold strains), influenza, measles, respiratory syncytial virus (RSV), Human metapneumovirus (HMPV), and bacteria such as M. tuberculosis, is a significant factor in the transmission of the infectious diseases they cause [1]. Mitigating the airborne spread of these pathogens is crucial for public health. Interventions such as increased air exchange, filtration, and ultraviolet germicidal irradiation (UVGI) aim to reduce the concentration of viable pathogens in the air, hence, reducing the risk of infection by reducing inhalational exposure [2]. Among these interventions, UVGI systems hold great promise for effective pathogen control, offering both significant efficacy and cost-effectiveness in terms of capital and operating expenses [3].

The effectiveness of an intervention may vary across different settings and might require numerical simulations and empirical testing. For example, an air cleaning system for a hospital might greatly benefit from design and optimization, whereas for non-critical environments such as a residential setting, such modeling and optimization would be impractical and cost prohibitive [4]. Numerical simulations, such as Computational Fluid Dynamics (CFD) and fluence rate field models (e.g., ray tracing), provide insights into the potential distribution and impact of UVGI within enclosed spaces. CFD is commonly used to simulate indoor air quality (IAQ) dynamics in the built environment, although its broad application in commercial building settings can be challenging [5,6].

Empirical testing, on the other hand, involves experiments using non-pathogenic challenge agents to quantify inactivation or physical separation under various operating conditions. Conventionally, such tests have been conducted in specialized environments like IAQ chambers. However, a critical challenge arises in translating test results from controlled environments to commercial building application settings. Currently, there is no suitable method that combines mimicking the particle size distribution of a human upper respiratory aerosol emission that can be used for onsite testing of UVGI system efficacy.

A limitation on effective application of the current methods for reducing human exposure to airborne pathogens in public spaces is the lack of a safe, single indicator that can represent the effectiveness of air purification devices, while taking into account the multiple mechanisms that affect pathogen decay rate. Given the inability to assess installed effectiveness, it is often difficult to justify the cost of air filtration and cleaning devices, such as filters or UVGI fixtures.

Respiratory aerosols can be generated from alveolar, bronchiolar, bronchial, laryngeal, and oral fluids [7,8]. Airborne pathogens from human emissions are released in these fluids, then they desiccate to a fraction of their initial size [9], depending on initial particle size and environmental conditions. Their concentration in air will diminish by gravitational deposition, natural inactivation, removal by filters, dilution by the introduction of clean outside air, inactivation by exposure to UV radiation, and other causes.

Various examples herein will be described with reference to UVGI equipment under test. Such UVGI equipment might be installed in an enclosed space or an unenclosed space and might be used to provide a germicidal effect as air, water, and/or material passes over, around, through, or by the UVGI equipment and is exposed to UV energy. In specific examples, an effectiveness of the UVGI equipment might be determined by a ratio of live or active pathogen or pathogen surrogate concentrations before UV exposure compared to concentrations after UV exposure.

In one application, a germicidal UV-C responsive aerosol tracer can be based on a DNA sequence and can provide for in-situ indoor air quality (IAQ) Analysis. The sequences can be provided as an aerosol or in a solid form, such as on a coupon or disposable substrate, and can be DNA or a chemical compound with known or determinable UV degradation characteristics. In some ways, DNA degradation in a test can be used to characterize DNA degradation in a pathogen or other pathogen-destroying effects of the application of UV light.

In examples herein, a germicidal verification system uses two or more tags that are not pathogenic and have differing UV sensitivities. In some embodiments, the tags comprise noncoding strings of nucleic acid base pairs, such as DNA sequences, RNA sequences, etc. The tags can be introduced into or upon the air, water, and/or material that is the matter to be sanitized. The tags can be introduced to the matter prior to UV exposure and then following the UV exposure can be tested. With the tags comprising noncoding strings of nucleic acid base pairs, some of those tags might be wholly or partially destroyed or degraded by the UV exposure or at least modified in ways that can be detected.

The tags might be tags that would be used in the VeriDART™ system developed by SafeTraces, Inc. (SafeTraces). Post-exposure detection of the tags might be as with the VeriDART™ system. By using two or more tags with differing UV sensitivities, changes in pathogen surrogate concentrations due to UV exposure can be distinguished from changes in concentrations due to other factors, such as air mixing and dilution, flow obstructions, and/or introduction of other materials.

As explained herein, a tag might include a primer portion, a tag identifying sequence, and a dose-responsive sequence. In a typical installation, at least two tags are used that have different dose-responsive sequences. The sensitivity of a tag to UV degradation might be controlled by selection of base pairs used in the dose-responsive sequences. The primer portions might be highly robust to UV exposure for case of identifying in a post-exposure test. Identification of tags might be done using DNA sequence amplification techniques.

The tags might be deployed in an aerosol form that can be mixed with air that is to be sanitized. The tags might be deployed in a solid form, such as being applied to coupons that can be attached to surfaces. For example, a coupon formed as a sheet of foil, paper, or other substrate, having a tag or tags attached thereto, possibly with an adhesive layer, might be applied to a surface in a food processing facility prior to UVGI exposure. Following the UVGI exposure, the coupon could be removed and the tag or tags thereon processed by DNA sequence amplification techniques.

By noting an original concentration of a first tag and an original concentration of a second tag, and measuring a post-exposure concentration of the first tag and a post-exposure concentration of the second tag, a UVGI effectiveness processing system can determine a germicidal effectiveness of the exposure by considering the original versus measured concentration of the first tag compared to the original versus measured concentration of the second tag. Given that the first tag and second tag have differing robustness to UV exposure, the differences in the changes of concentration could be used as a measure of UVGI effectiveness.

In some situations, it might be useful to measure how drying the air is in the environment under test and tune the aerosols accordingly. For example, if a large room is to be tested and the air is very dry, larger aerosol droplets might be called for so that not all of the tagged aerosol immediately dries and falls to the floor.

Depending on the application, particular levels of robustness might be required. For example, where a very high UV exposure is expected, each tag that is used might have a high robustness and where low UV exposure is expected, a tag might be made to be very sensitive to UV exposure. The robustness level of a tag's dose-responsive sequence might be varied and selected by particular selection of a pattern elements used for the tag. In a specific example, the elements of tags are nucleic acid sequences and the nucleic acid sequences that are selected for a given tag are selected based on the nucleic acid sequences' sensitivity to certain radiation, such as UV radiation, and therefore tags are more robust or more easily degraded depending on which pattern elements are selected and used for those tags.

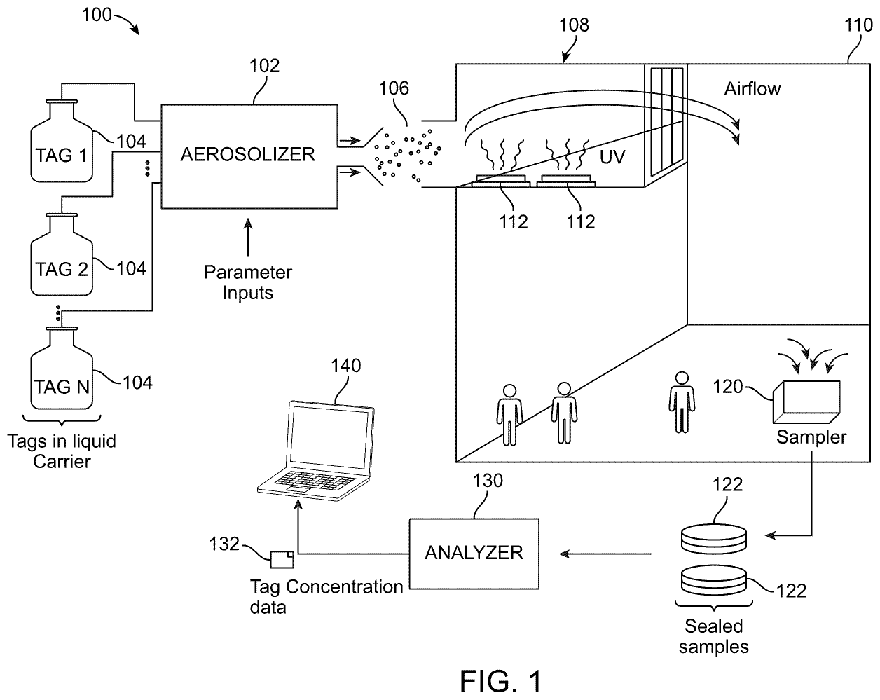

FIG. 1 is a diagram of a germicidal effectiveness testing configuration, according to various embodiments. FIG. 1 illustrates an example of an implementation of a UVGI effectiveness processing system in an environment 100. As shown there, an aerosolizer 102 takes in tags, which might be stored in containers 104, which might be tanks, vials, bags, etc. that feed into aerosolizer 102, which then generates an aerosol output 106 that is directed into a UV-based purification system 108. UV-based purification system 108 is shown as an integral part of a room 110 but other arrangements are possible.

As the airflow through UV-based purification system 108 is purified and exposed to UV radiation from UV lamps 112, the air passes into room 110 and might circulate. An air sampler 120 can draw in air from room 110, using a fan, passively collecting air, or some other process to sample the air. The collected samples might be sealed into containers 122 for transport to a different location where an analyzer 130 can analyze the collected samples. In other embodiments, analyzer 130 might be on-site proximate to air sampler 120.

Operations of an analyzer such as analyzer 130 are described herein and an output of analyzer 130 might be data records or messages containing tag concentration data 132. Tag concentration data 132 might be provided to a computer 140 that processes the data to compute an inferred UV germicidal effectiveness based on tag concentration data 132 and possibly other parameters.

Transmission of respiratory pathogens occurs primarily in indoor settings. Engineering interventions that are known to reduce the risk of transmission of respiratory pathogens include increases in outdoor air introduction, filtration, and inactivation by technologies including ultraviolet germicidal irradiation (UVGI). However, testing and validation of these interventions is challenging, particularly in actual applications. As explained herein, by introducing an aerosol tracer system with taggants that comprise DNA sequences, quantitative characterization of the performance of indoor air cleaning systems can be determined, in particular when those indoor air cleaning systems affect DNA sequences and also when those indoor air cleaning systems affect different DNA sequences differently. In an approach described herein, two DNA tags, one designed to be relatively UV-resistant, and another designed to be relatively UV-sensitive, are used as tracers to assess air quality changes related to filtration and ventilation systems and the unique contributions of UVGI fixtures in various built environments. In controlled UV exposure experiments of DNA-tagged tracers on foil coupons, aerosolization studies in a test chamber, and in as commercial building conference room, the effectiveness can be quantized. DNA tags can be analyzed using digital PCR methodologies, providing insights at the point of sampling into the effects of complex airflow dynamics. Additionally, ways to scale DNA results to MS2 bacteriophage concentrations are proposed. As explained, a quantitative understanding can be obtained of interactions between UV-sensitive aerosols and a built environment, with implications for precision environmental monitoring, measuring UVGI fixture impact in field settings, and addressing limitations in current technologies for assessing UVGI disinfection efficacy.

FIG. 1 illustrates the case of using N tags for N=2, 3, . . . and in examples below, two tags are used in an apparatus and process for evaluating an environment and/or equipment or processing deployed in that environment as to germicidal effectiveness of the equipment or processing, such as when UV irradiation is used as a germicide. In some implementations, a single tag might be sufficient.

In an example approach, two nucleic acid base tags are deployed in the environment and samples are collected. One tag is relatively insensitive to UV irradiation while another is relatively (relative to the first tag) sensitive to UV irradiation. Analyzing collected samples can determine a baseline of how an aerosol behaves in a building environment without UV degradation. The sensitive tag that has a dose response degradation to UV exposure can be used to measure how well a germicidal effect is of a UV lamp treatment. Nucleic acid concentration measurements can be mapped to an estimated microbial germicidal dose response. Correlating sampled nucleic acid concentrations with a measured amount of UV exposure can provide an indication of germicidal effectiveness. For example, if a room's airflow is exposed to X mJ/cm² of UV radiation over a time, T, and sampled nucleic acid concentrations indicate a ratio, R, of degradation of a sensitive tag relative to an insensitive tag, that might correspond (perhaps based on empirical studies performed in controlled environments) to an estimate of the UV treatment having killed Y % of a pathogen P.

In other embodiments, tag elements might be other than nucleic acids and similar approaches might be used for such tags.

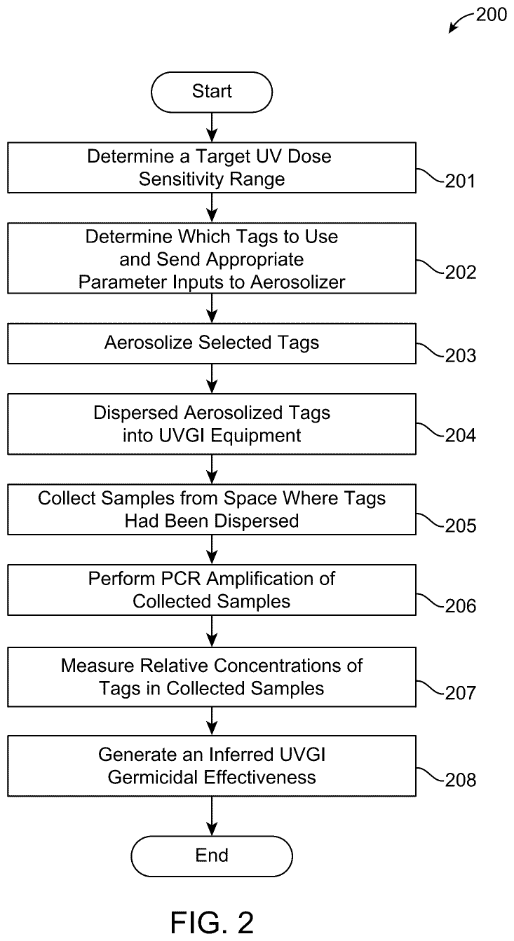

FIG. 2 is a block diagram 200 of a process for determining an inferred UV germicidal effectiveness of UVGI equipment. As shown there, process might begin with step 201, wherein a system controller-possibly controlled by a system operator-determines a target UV dose sensitivity range. Then, at step 202, the system controller determines which tags to use and would send the appropriate parameter inputs to an aerosolizer (such as aerosolizer 102 shown in FIG. 1). In some embodiments, the system controller might be integrated in with the aerosolizer. Next, at step 203, the selected tags are aerosolized. At step 204, the aerosolized tags are dispersed into a UV sterilizer or other UVGI equipment.

Next at step 205, samples are collected from the space where air into which the tags had been dispersed and that has passed through the UVGI equipment. Given that the tags are noncoding nucleic acid sequences, and nonpathogenic, circulation in an occupied space should not be problematic. At step 206, an analyzer performs PCR amplification of the collected samples. At step 207, the analyzer measures relative concentrations of tags in the collected samples. With suitable selection of UV dose sensitivity via selection of the dose-responsive sequences of tags, degradation of the dose-responsive sequences as a result of UV exposure would be expected and can be used as a proxy for germicidal effects of such UV exposure. Relative degradation of the dose-responsive sequences as between two tags can be determined from the measured relative concentrations in the collected samples.

For example, consider the case where a first tag is highly robust to UV irradiation and a second tag is easily damaged by UV radiation. If equal quantities of the two tags are introduced into the UVGI equipment and equally mixed in the occupied space, it might be assumed that differences in concentration of the two tags in a collected sample is due to UV irradiation. It may be that physical concentration of tag material in the collected sample of each of the tags is the same but because of the modification or destruction of the nucleic acid sequence in the more sensitive tag, the more sensitive tag would not be amplified is much or would not be recognized as such if amplified.

As a next step 208 in process 200, the analyzer might-possibly in combination with other collected data—infer a UV germicidal effectiveness.

In some implementations, the tags are in aerosol form, whereas in other implementations, the tags of might be deployed on coupons such as foil coupons. With an aerosol implementation, UV systems in buildings, such as HVAC system ducts, wall-mounted UV room purification systems, etc. can be assessed. Coupons can be used to assess direct UV lamp effectiveness, such as hood UV systems in biological cabinets, spaces within UV purifications systems, irradiation of food-bearing surfaces, etc.

In some implementations, a single sensitive tag is deployed and the estimated germicidal dose response might be based on a ratio of the concentration of the single sensitive tag placed into the environment under test divided by the concentration of the single sensitive tag collected in a sample.

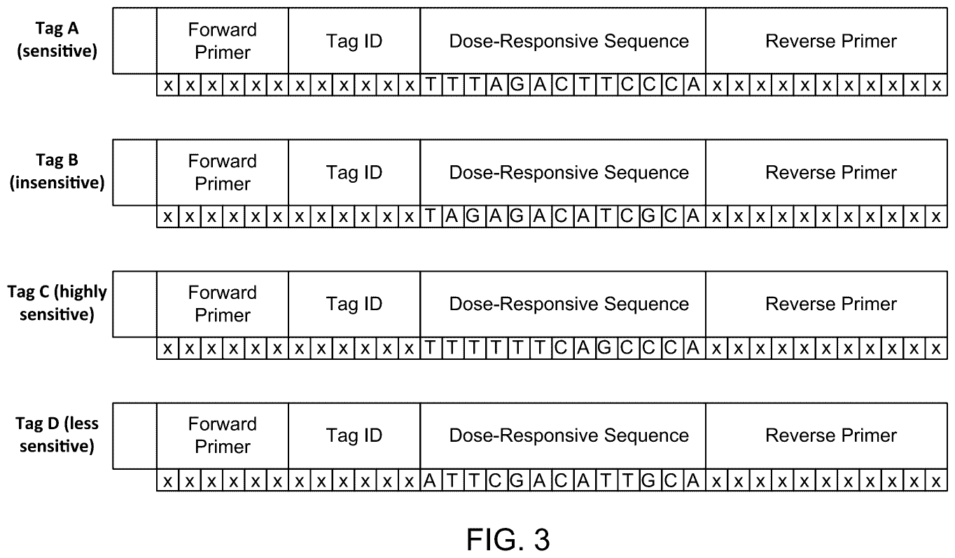

FIG. 3 illustrates various tags that might be used in a testing process, according to various embodiments. As illustrated, each tag includes a forward primer, a tag ID, a dose-responsive sequence, and a reverse primer. The forward primer and reverse primer might be some nucleic acid sequences to assist with processing collected tags. The tag ID might be a nucleic acid sequence that uniquely identifies the tag and might come into play when multiple tags are released at different locations within an environment and intermixed. It should be understood that the symbols “x” in FIG. 3 refer to some particular, albeit unspecified herein, nucleic acid sequence.

As illustrated, Tag A is designated as being a relatively sensitive tag given that its dose-responsive sequence is “TTTAGAC” followed by “TTCCCA” with a number of adjacent thymine pairs. Exposure to UV light can cause degradation of Tag A's dose-responsive sequence by producing pyrimidine dimers such as thymine dimers between adjacent thymine bases or cytosine dimers between adjacent cytosine bases. Where appropriate, RNA chains might be used for tags, with similar UV exposure effects. Using PCR on collected samples, the tag sequences can be detected. Thus, PCR can detect the existence of certain DNA sequences, and with that, can detect the absence, or relative absence, of other DNA sequences. As a result, the detection of only a portion of a tag that is known to be present, as opposed to detecting the entire tag, can be attributed to UV exposure effects. The UV exposure need not destroy all of a tag, just enough of the tag to alter the PCR results. That alteration might be due to the PCR process being disrupted by a tag's degradation such as the binding of nucleotides to other nucleotides, to themselves, or other UV-driven alterations in the tag.

By contrast, Tag B, which is indicated to be a relatively insensitive tag, has a dose-responsive sequence of “TAGAGAC” followed by “ATCGCA” that does not include any matching adjacent base pairs. In some testing, it might be useful to have varying degrees of sensitivity, such as Tag C—a highly sensitive tag—that has a dose-responsive sequence of “TTTTTTC” followed by “AGCCCA” and Tag D—a sensitive, but less sensitive tag—that has a dose-responsive sequence of “ATTCGAC” followed by “ATTGCA”. Thus, based on the environment, the levels of UV exposure, sampling rates, etc., the tags to be used can be selected based on their sensitivity. In some testing approaches, overly sensitive tags that completely degrade and overly insensitive tags that experience no degradation might be less preferred over tags that have a degree of degradation that is between those two extremes.

In one specific implementation, a tag might comprise a sequence corresponding to a 6-4 photoproduct, such as (6-4 pyrimidine-pyrimidone or 6-4 pyrimidine-pyrimidinone). With such a tag, there is an alternate dimer consisting of a single covalent bond between the carbon at the 6 position of one ring and carbon at the 4 position of the ring on the next base. This provides a sequence TCCCTT which has been shown to contribute to the 6-4 photoproduct which can block DNA replication, and hence PCR.

When exposed to UV, a contiguous set of T residues in nucleic acid might result in crosslinks of the T bases to each other that would impede amplification by PCR that can be detected when sampling. A PCR process might amplify the primers and tag IDs but not the dose-responsive sequences if the latter are destroyed by the UV light. Using two (or more) tags can allow for accounting for aerosol clearance by room air conditions so that dilution (or concentration) due to airflow is not attributed to UV exposure degradation and can thus provide, at least a proxy, measurement of how much microbial germicidal effect the UV exposure is providing.

Using DNA tags as a surrogate for pathogens to make assessments about indoor air quality and pathogen removal by UV technology allows for data gathering in live occupied environments. The teachings herein can be used with a field deployable qPCR system or samples can be sent to a lab for analysis to measure tag concentration changes.

By measuring degradation of sampled tags collected from an environment wherein a UV exposure treatment has been supplied, effectiveness of the UV exposure treatment UV exposure treatment in situ can be estimated. The collection could be done using techniques used for airflow, ventilation, and filtration for air movement measurements as might be done using equipment supplied by SafeTraces.

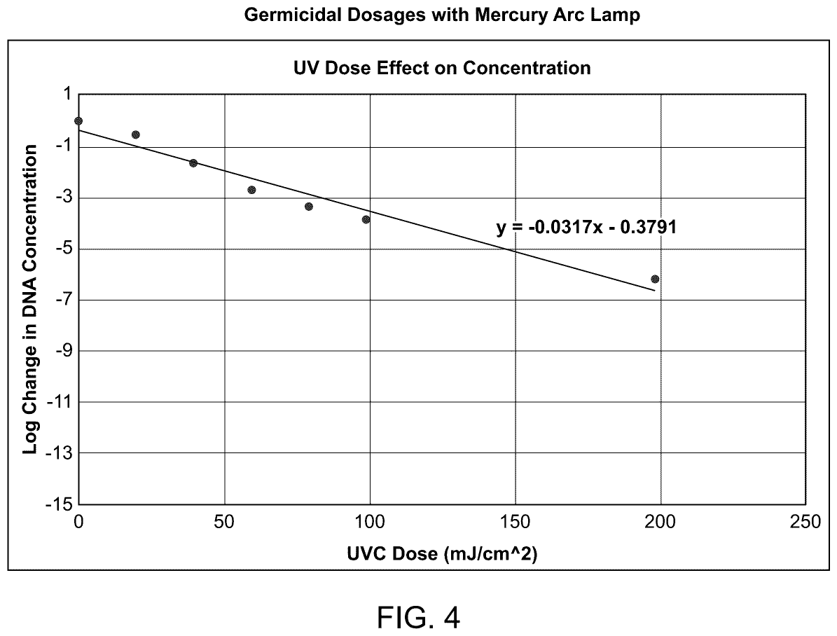

FIG. 4 is a plot, according to various embodiments.

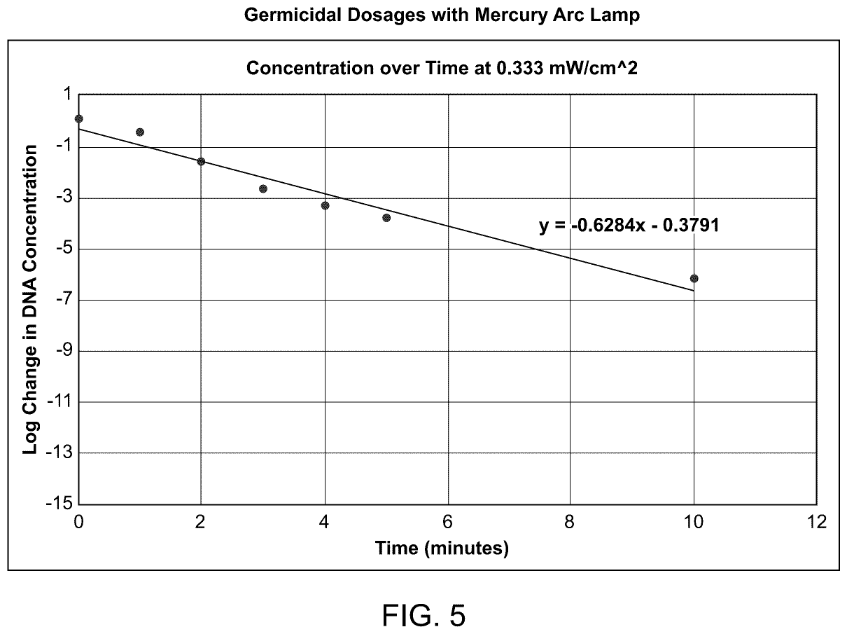

FIG. 5 is a plot, according to various embodiments.

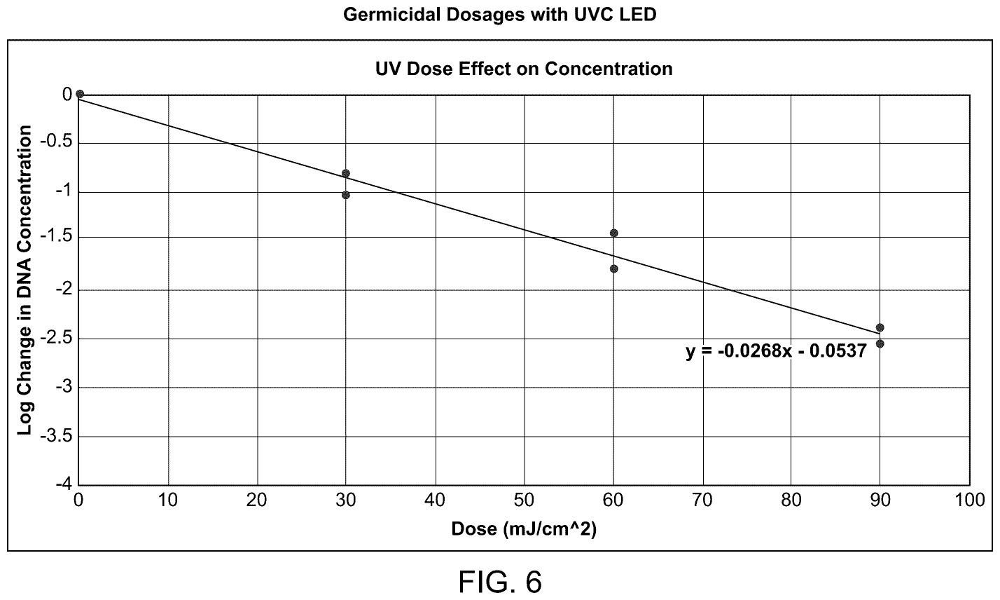

FIG. 6 is a plot, according to various embodiments.

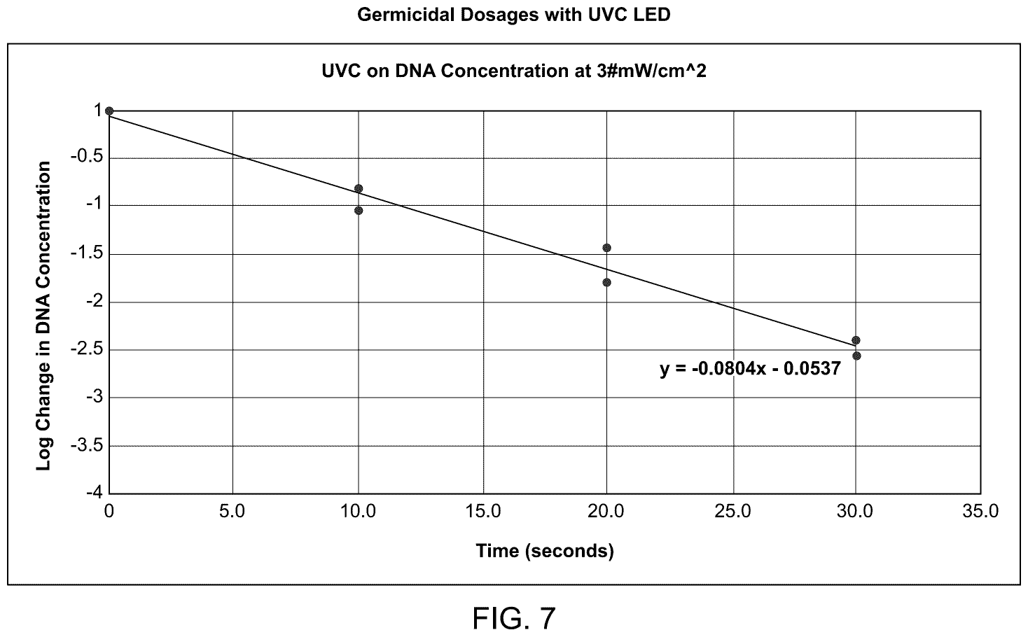

FIG. 7 is a plot, according to various embodiments.

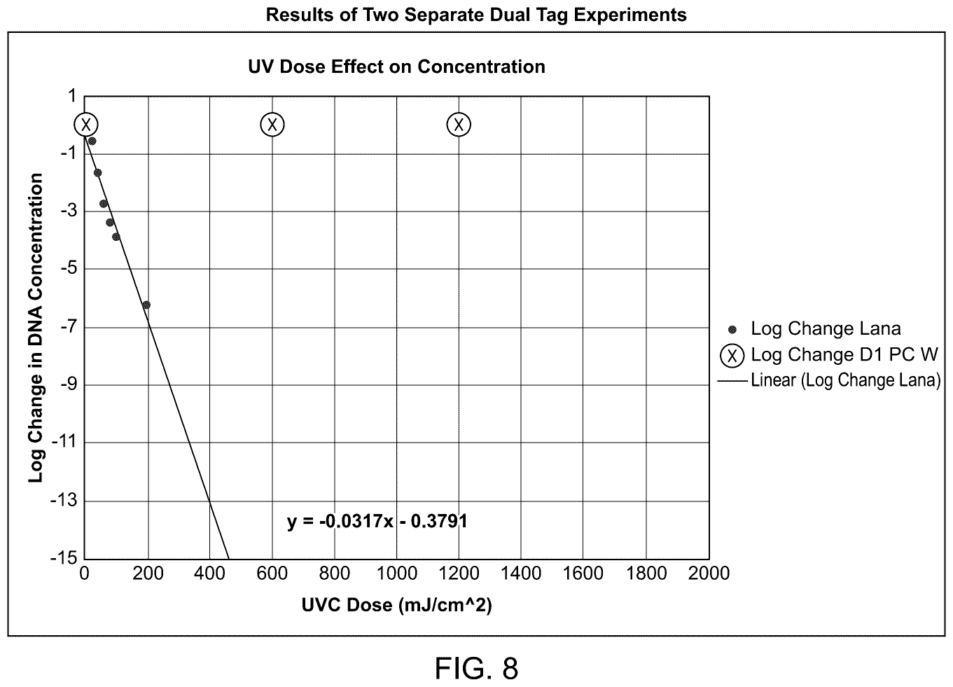

FIG. 8 is a plot, according to various embodiments.

The plots of FIGS. 4-8 indicate various ranges for doses and changes in DNA concentration. As explained herein, the sensitivity of the tags used might vary and might be changed depending on the pathogen of concern. For example, E. Coli might require a UV radiation germicidal dosage of 1.9 mJ/cm², Legionella pnemophila might require 0.9-3.1 mJ/cm², while SV40 might require 92.8 mJ/cm². Thus, testing for SV40 germicidal effect, given that airflow is exposed to higher levels of UV, would indicate for the use of less sensitive tags to avoid the sample measurements indicating that all of the tag material is degraded.

Tag selection might take into account germicidal dosages. Effectiveness of a treatment might be measured by what level of log₁₀ inactivation occurs for a given dose and the given dose might be specified in terms of a UV band or wavelength range, an intensity level, and an amount of time. The intensity used might be a function of the equipment used. For example, a mercury hood might provide 0.33 mW/cm² of broad-spectrum UV and an “R” UV producer might provide 0.2 mW/cm² of UVC.

Intensity might be measured in milliwatts per square centimeter (mW/cm²) and a dose (e.g., an intensity over a period of time) might be measured in millijoules per square centimeter (mJ/cm²).

As explained herein, a UV-sensitive aerosol tracer system using DNA or a chemical compound as a tracer molecule can offer precision environmental monitoring capabilities. The system might employ two distinct DNA tracers, one selected for its robustness upon being exposed to UV radiation and another one selected for being highly UV-sensitive, to assess air quality alterations related to filtration and ventilation systems and/or UV fixtures in various built environments. Some experimental analyses and results are provided hereinbelow, including controlled UV exposure and real-world aerosolization. These can show a system's operation and usefulness for optimizing indoor air quality and safety. The system can provide for environmental monitoring, providing valuable insights into interactions between UV-sensitive aerosols and built environment systems, with implications for public health and safety.

### Test Results

As the test data herein shows, the described system can enable quantification of DNA-tagged aerosols captured on air filters, facilitating their analysis through digital PCR methodologies. UV-sensitive aerosol technology can aid in the understanding of intricate airflow dynamics within built environments equipped with UV fixtures. In an embodiment, the system comprises two (or more) distinct DNA tracers, wherein one is designed or selected to remain relatively unaffected by UV radiation and the other displaying a high degree of UV sensitivity. The former provides data regarding alterations in air quality due to filtration and ventilation systems within the workspace under scrutiny. Meanwhile, the UV-sensitive DNA tracer offers additional insights into the unique contributions of UV fixtures in conjunction with ventilation and filtration strategies. A differential analysis between these tracers can unveil the precise impact of UV fixtures, a parameter that can be scaled to gauge the logarithmic reduction of viral aerosols achieved through the deployment of UV fixtures.

The data show here was collected from tests of an UV-sensitive aerosol tracer system using foil coupons with a DNA-tagged tracer solution containing both the UV-sensitive and UV-resistant tags as well as aerosols.

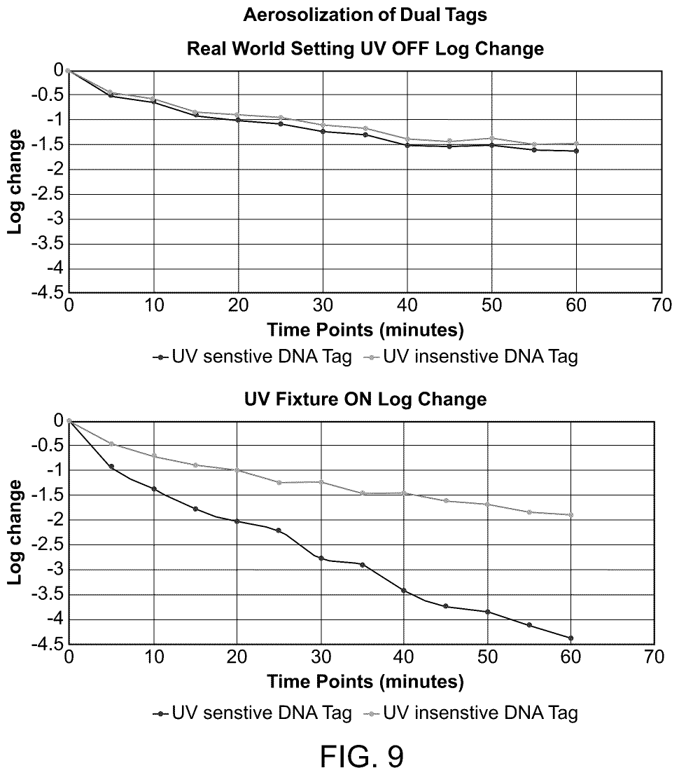

FIG. 9 illustrates test results using aerosolized DNA tags, in two plots. Each of the plots are of log change (ranging from −4.5 to 0) as a function of time (in minutes, plotted from 0 to 60 minutes). The left plot is in the real-world setting with the UV device off. The lower curve and set of points are for the UV sensitive DNA tag and the upper curve and set of points is for the UV insensitive DNA tag. The difference between the two curves is small. The right plot is in the real-world setting with the UV fixture on for the environment under test, which could be an occupied volume or a to-be-occupied volume. The lower curve and set of points are for the UV sensitive DNA tag and the upper curve and set of points is for the UV insensitive DNA tag. In the example shown, there is more than a 2*log₁₀ change (e.g., more than 100× reduction) over one hour of air sampling. The difference on the right is much greater than on the left, illustrating the measurability of UV fixture effects on DNA tags of varying sensitivities, which could be used as a measure of UV fixture effects on pathogens and/or pathogen surrogates.

In a test to generate such data, two tags (UV insensitive and UV sensitive tags) can be aerosolized and exposed to UV light sources in a commercial space. The aerosol can then be collected on an air sampler filter and processed, such as by counting or estimating a number of nondegraded DNA molecules under the on and off conditions. The degradation might be the result of the UV light damaging the DNA of the tags such as that when PCR is done, those damaged tags do not appear to be replicated as would have occurred under PCR with undamaged DNA.

Whether done using coupons or aerosols, whether done with DNA tags or other compounds on tags, data can be gathered in a controlled environment away from the environments to be tested and that data can involve the use of the tags in combination with pathogen simulants such as MS2 phages, and then that data can be used for scaling results of tags that are exposed in the environments to be tested.

For the foil coupon tests, the coupons were coated with the tracer solution, air-dried and subsequently exposed to a precisely collimated UV light source centered at three distinct wavelengths: 222 nm, 254 nm, and 282 nm. The 222 nm light might be generated using an optically-filtered KrCl excimer UV lamp. The 254 nm light might be generated using a low-pressure Hg UV lamp. The 282 nm light might be generated using a UV LED lamp. The optically-filtered KrCl lamp might emit peak irradiance at 222 nm with a full width at half maximum (FWHM) bandwidth of roughly 2 nm. The low-pressure Hg lamp can be essentially monochromatic at 254 nm, with a FWHM bandwidth of less than 1 nm. The UV LEDs used in experiments described herein have an emission peak at about 282 nm, with a FWHM bandwidth of roughly 10 nm. All three sources had minor emission lines at longer wavelengths. Other lamps with other characteristics might be used instead.

The data obtained from these coupon-based experiments can provide a basis for subsequent aerosolization studies. In a controlled test chamber environment characterized by zero air changes per hour, DNA tags were aerosolized simultaneously with MS2 phage aerosols. Performing aerosolization experiments at wavelengths of 222 nm, 254 nm, and 282 nm indicated interactions between the dual DNA tags and UV-C fixtures in a controlled, yet dynamic, experimental setting. Further simulating a real-world application, the dual DNA tags were subsequently aerosolized within an authentic workspace environment. This real-world scenario provided evidence of practical implications of the UV-sensitive aerosol tracer system and gauging its effectiveness under dynamic conditions of a live workspace. Other wavelengths might be used.

Simultaneous release of the MS2 phage with the DNA tracer can allow for scaling of the UV-sensitive DNA tag inactivation to the MS2 phage inactivation. MS2 bacteriophage is commonly studied in aerosolization experiments as it is relatively innocuous and has been used as a surrogate for, and correlated with, known infectious disease airborne pathogens. Scaling with the commonly used microbiological virus, MS2, can provide a direct correlation of DNA tag to bacteriophage reduction and ultimately to understanding the effectiveness of UV fixtures in real-world settings on airborne pathogens.

For example, a known quantity of a tracer can be introduced into an environment and subsequently collected. A comparison of the quantity released and the quantity collected, using the UV-insensitive DNA tag (or with the UV system turned off) can provide a measure of air mixing and a baseline value. A comparison of the (quantity collected/quantity released) ratios for the UV-insensitive DNA tag and the UV-sensitive DNA tag upon UV exposure can be recorded in an environment under test. Scaling from those ratios to estimated effects on MS2 phages in the environment under test can be done using results of measurements in test chambers with UV exposure of the DNA tags and MS2 phages. Scaling further to estimated effects of UV on target pathogens in the environment under test (without having to introduce those targe pathogens into the environment under test) can be done using known relationships and correlations between MS2 phages and the target pathogen.

One test method comprised the steps of:
1) Foil Coupon Preparation and Irradiation
2) DNA Tag Aerosolization and UV Exposure in Test Chamber
3) Sample Collection and Analysis
4) Sample Processing and DNA Extraction
5) Dilution for Quantitative Analysis
6) Analytical Method: Droplet Digital Polymerase Chain Reaction (ddPCR)

### 1. Foil Coupon Preparation and Irradiation

Foil coupons were employed as a platform for irradiating dried DNA tags using a ddPCR (Droplet Digital Polymerase Chain Reaction) approach. DNA, tagged with a UV-sensitive compound, was spotted onto the foils in 50 μL volumes and allowed to dry completely. Subsequently, these dried DNA spots were subjected to UV light exposure at wavelengths of 222 nm, 254 nm, and 282 nm, with energy doses ranging up to 100 mJ/cm².

### 2. DNA Tag Aerosolization and UV Exposure in Test Chamber

DNA-tagged tracer samples were aerosolized within a controlled test chamber spanning 1050 cubic feet (ft³). A variety of commercial UV fixtures, including an Aeromed Lexus 2.1™ model fixture, a FarUV Krypton™ model fixture, a Bolb Suvos™ LED model fixture, and a Bolb Troffer™ model fixture, were the UV light sources. The Aeromed Lexus 2.1™ model fixture is an upper room germicidal UV device emitting at 254 nm, the FarUV Krypton™ model fixture is a whole room irradiation device emitting at 222 nm, the Bolb Suvos™ LED model fixture is an LED device for unoccupied space sterilization at 275 nm, and the Bolb Toffer™ model fixture is an enclosed LED UV at 275 nm in an air troffer with an internal fan at 160 cfm.

For aerosolization, 5 mL of the DNA-tagged tracer was simultaneously aerosolized with 5 mL of MS2 phage in PBS (Phosphate Buffered Saline) and soy tryptophan broth. Two distinct nebulizers provided by SafeTraces were used for this purpose. Samples were systematically collected every five minutes over a one-hour period within the test chamber.

### 3. Sample Collection and Analysis

Airborne samples were collected on Sterlitech™ 1-micron cassette filters, integrated into SafeTraces' air samplers. This allowed for precise and controlled sample collection, ensuring the retrieval of airborne particles containing the DNA tracer. Subsequent analysis was conducted to assess the efficacy of UV exposure and its impact on the DNA-tagged tracer under the diverse conditions tested.

This methodological approach aimed to comprehensively investigate the impact of UV exposure on the DNA tracer, both on solid surfaces and in aerosolized form within a controlled environment.

### 4. Sample Processing and DNA Extraction

Following exposure and aerosolization experiments, the DNA tags from the foil coupons were extracted for subsequent analysis. Each foil coupon was processed in 50 mL conical tubes using 500 μL of IDTE buffer at pH 8.0, but other preparations are possible. The buffer was supplemented with 100 mg of poly-A per liter, for efficient DNA recovery and stability.

Simultaneously, the Sterlitech™ 1-micron (AE2500) cassette filters integrated into Safe Traces air samplers were subjected to DNA extraction using the same buffer composition used for the foil coupons. The extraction process involved the addition of 500 μL of the buffer, followed by centrifugation to collect the DNA-containing supernatant.

### 5. Dilution for Quantitative Analysis

Upon completion of extraction, the extracted DNA samples, both from the foil coupons and air sampler cassettes, underwent dilution. This step can be done to ensure the samples fell within the quantitative range of the particular ddPCR system in use. Dilution factors ranged between 1 and 500 times, allowing for accurate and precise quantification of the DNA tags.

The comprehensive sample processing and dilution steps were implemented, in part, to guarantee that the subsequent ddPCR analysis would yield reliable quantitative results, enabling a detailed assessment of the impact of UV exposure on the DNA tags under various experimental conditions.

6. Analytical Method: Droplet Digital Polymerase Chain Reaction (ddPCR)

To elucidate the impact of UV exposure on the DNA tags, a highly sensitive and quantitative approach was employed-Droplet Digital Polymerase Chain Reaction (ddPCR).

This technique allows for the precise quantification of specific DNA sequences. Two TaqMan™ probes, each targeting either the UV-insensitive tag or the UV-sensitive tag, were utilized. These probes were labeled with distinct fluorophores-HEX for the UV-insensitive tag and FAM for the UV-sensitive tag, enabling discrimination between the two targets. Other labels could have been used.

The TaqMan probes could use a HEX-labeled TaqMan Probe for the UV-Insensitive Tag and a FAM-labeled TaqMan Probe for the UV-Sensitive Tag. To facilitate the ddPCR analysis, the following consumables were utilized: (a) ddPCR Supermix for Probes, (b) Droplet Generation Oil for Probes, (3) ddPCR 8-Well Cartridges, and (4) ddPCR Gasket Seal for 8-Well Cartridges.

Each diluted DNA sample, extracted from foil coupons and air sampler cassettes, was subjected to ddPCR analysis using the specified TaqMan probes. The amplification reactions were performed on a ddPCR thermal cycler, and the resulting droplets were analyzed using the QX200 Droplet Reader. This method allowed for the quantification of both UV-sensitive and UV-insensitive tags, providing a robust means to assess the effectiveness of UV exposure in altering the DNA tags' integrity. The utilization of specific TaqMan probes and associated consumables provided accuracy and reliability of the ddPCR-based analysis.

### Specific Test Results and Data

### Experiment 1: DNA Tag on Foil Coupon with Exposure to Various Wavelengths and Doses of UV

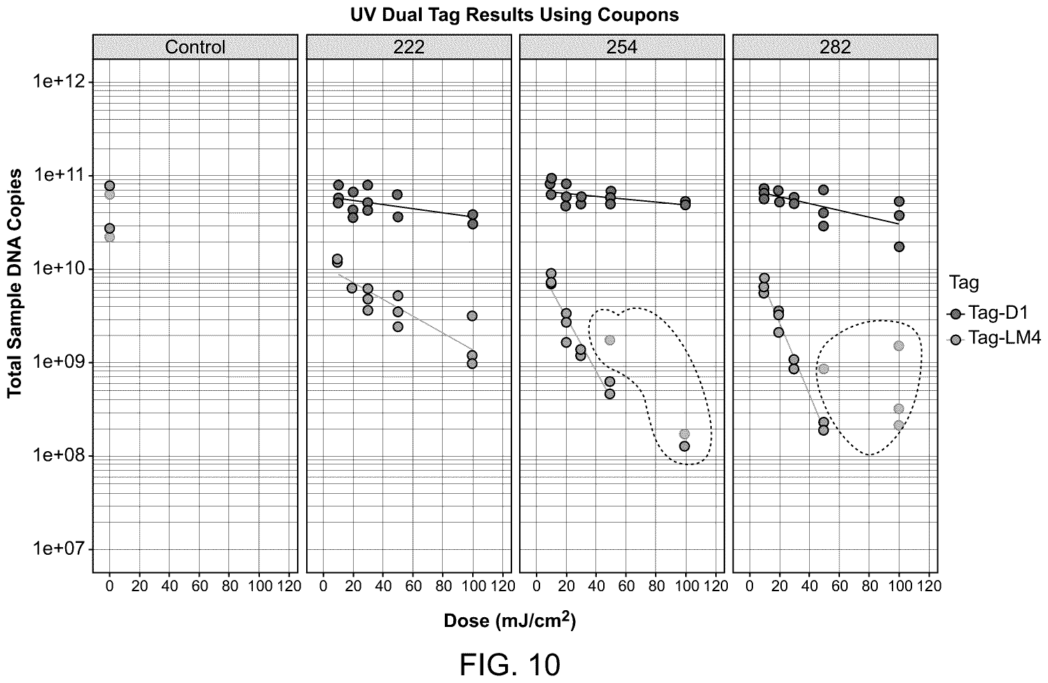

FIG. 10 illustrates UV Exposure Effects on DNA Tags on Coupons. FIG. 10 shows plots of results of UV exposure experiments on foil coupons, focusing on the UV-sensitive and insensitive tags. The investigation covered UV wavelengths of with doses ranging from 0 to 100 mJ/cm². The first, left-most, segment of FIG. 10 shows the results for a control, the second segment for 222 nm exposure, the third for 254 nm exposure, and the fourth (right-most) for 282 nm exposure. The y-axis represents the total DNA copies measured using ddPCR, while the x-axis depicts the dose in mJ/cm².

In each of the three exposed plots shown, the upper dots clustered around the upper lines represent the UV-insensitive or resistant tag, while the lower dots clustered around the lower lines represent the UV-sensitive tag. The lines can be determined by fitting to the respective points. Some of the data points are excluded as being a non-linear response. Those are shown within the dotted line regions in the 254 nm run and the 282 nm run.

FIG. 10 reveals distinct patterns in the dose response of the UV-sensitive tag (Tag-LM4; the lower groups) at different wavelengths. At 222 nm, a linear dose response is observed, showing a reduction in DNA copies with increasing UV dose up to 100 mJ/cm². For 254 nm and 282 nm, the UV-sensitive tag demonstrates a linear response up to around 40 mJ/cm², beyond which the reduction in signal becomes non-linear (the dotted line regions). Notably, the UV-insensitive or resistant tag exhibits significantly less reduction in signal across all UV wavelengths and doses (the upper dots and lines, Tag-D1), indicating its resistance to the deleterious effects of UV exposure.

### Experiment 2: UV Exposure Effects on DNA Tags Decay Rates on Coupons

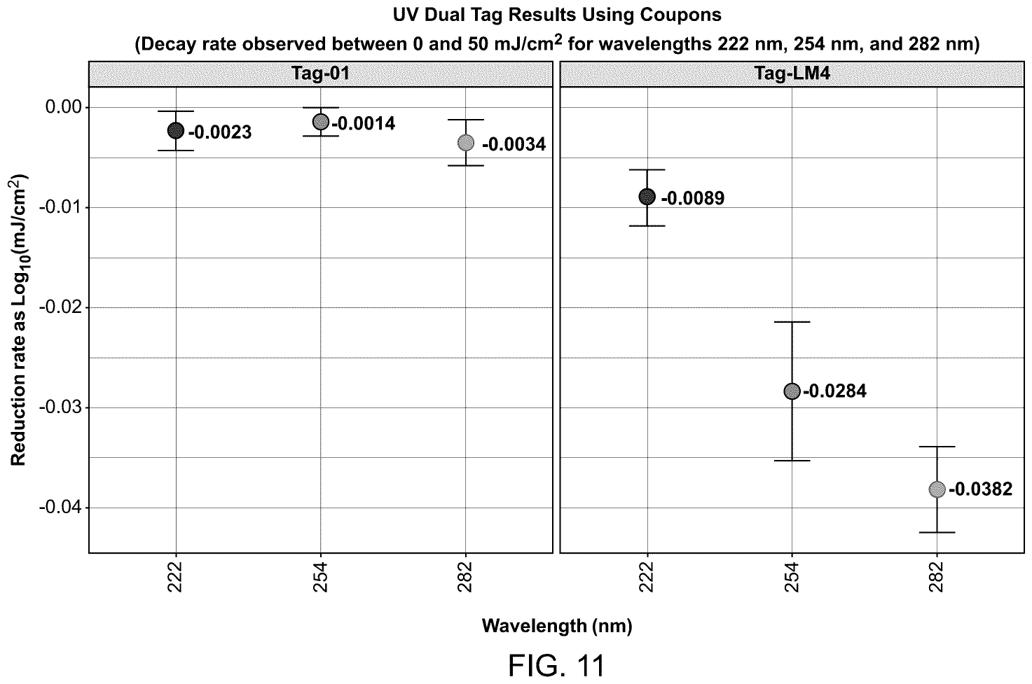

FIG. 11 illustrates decay rates as a function of UV irradiance at wavelengths of 222 nm (leftmost dot on each of the two segments of FIG. 11, 254 nm (center dot of each segment), and 282 nm (rightmost) with respect to UV-resistant Tag-D1 (left segment of FIG. 11) and UV-sensitive Tag-LM4 (right segment of FIG. 11) dried on foil coupons. As illustrated there, the log₁₀ reduction rates for the UV-resistant Tag-D1 are (−0.0023, −0.0014, −0.0034) for the 222 nm, 254 nm, and 282 nm exposures respectively, while the log₁₀ reduction rates for the UV-sensitive Tag-LM4 are (−0.0089, −0.0284, −0.0382).

Using test results such as these, differential impact of UV exposure on the amplification of DNA copies can be determined. The observed reduction in DNA copies on a UV dose basis can provide a measure of efficacy of UV in diminishing the amplifiable DNA content, with implications for applications involving DNA integrity assessment in UV-exposed environments.

### Experiment 3: DNA Tag and Bacteriophage MS2 Dual Aerosolization in ASHRAE 241 Compliant Test Chamber at 0 ACH with UV Test Fixtures

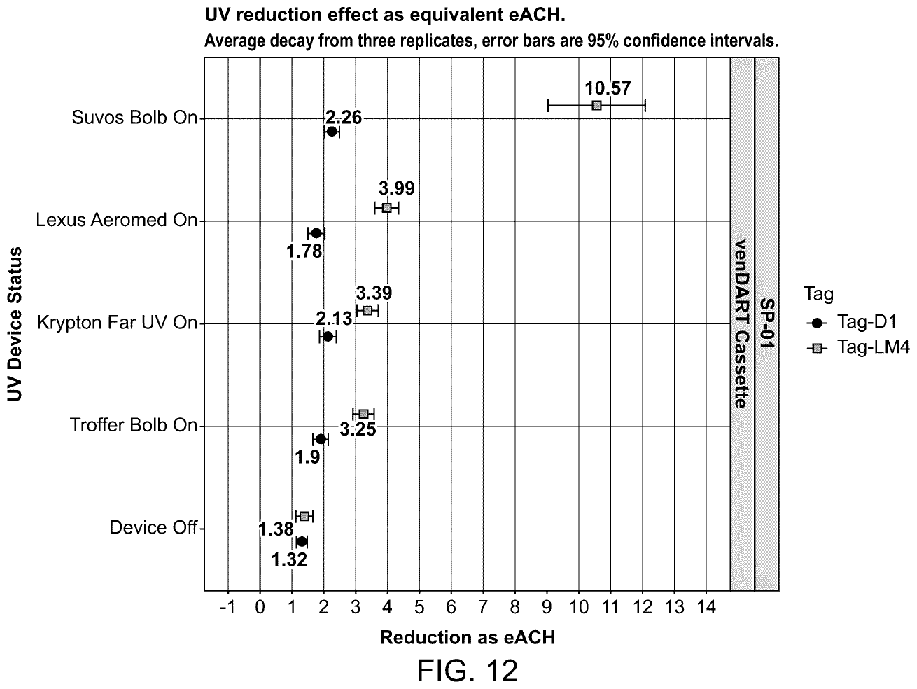

FIG. 12 shows plots of UV effects on aerosolized tags expressed as equivalent air changes per hour (eACH), calculated on a per UV fixture basis with respect to the dose response decay of acrosolized UV-sensitive Tag-LM4 and UV-resistant Tag-D1 in a 1050 ft³ test chamber with 0 actual ACH. As expected, with the device off, the eACH for both tags are similar (1.32 for the UV-resistant Tag-D1 and 1.38 for the UV-sensitive Tag-LM4) and with the devices on, the eACH for the UV-sensitive Tag-LM4 is greater than for the UV-resistant Tag-D1. Other experiments are possible in a test environment that has a non-zero ACH.

### Experiment 4: UV Effects on Bacteriophage MS2 as eACH

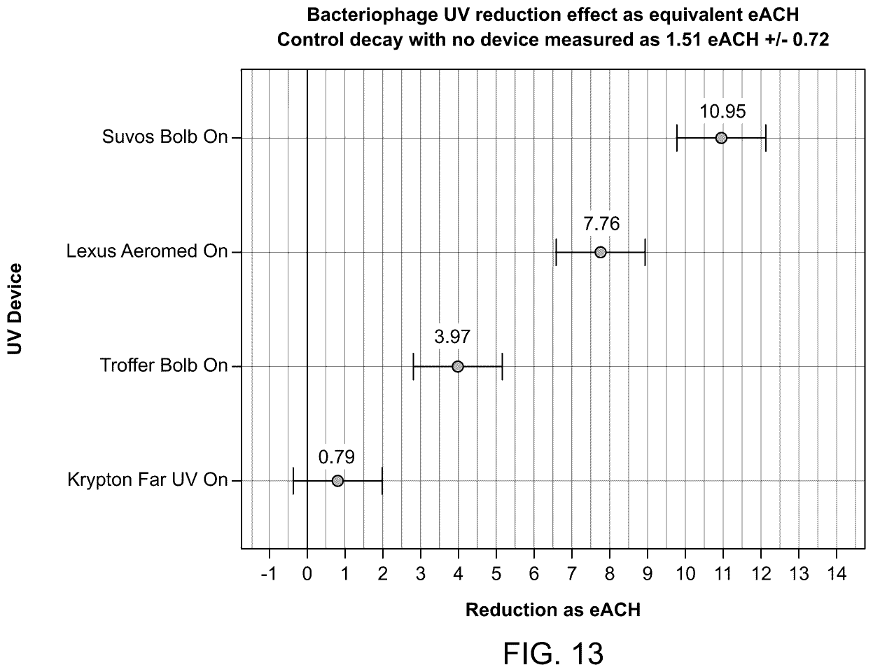

FIG. 13 shows plots of UV effects on bacteriophage MS2 as eACH. As shown there, results of the various UV fixtures under study with aerosolized bacteriophage MS2 in a 1050 ft³ test chamber with 0 ACH are expressed in eACH. The background decay of the bacteriophage in the chamber was subtracted to normalize the values of natural decay of the bacteriophage due to deposition of the aerosol over a one-hour time period. A control decay with no device measured as 1.51 eACH+/−0.72.

### Tracer Scaling

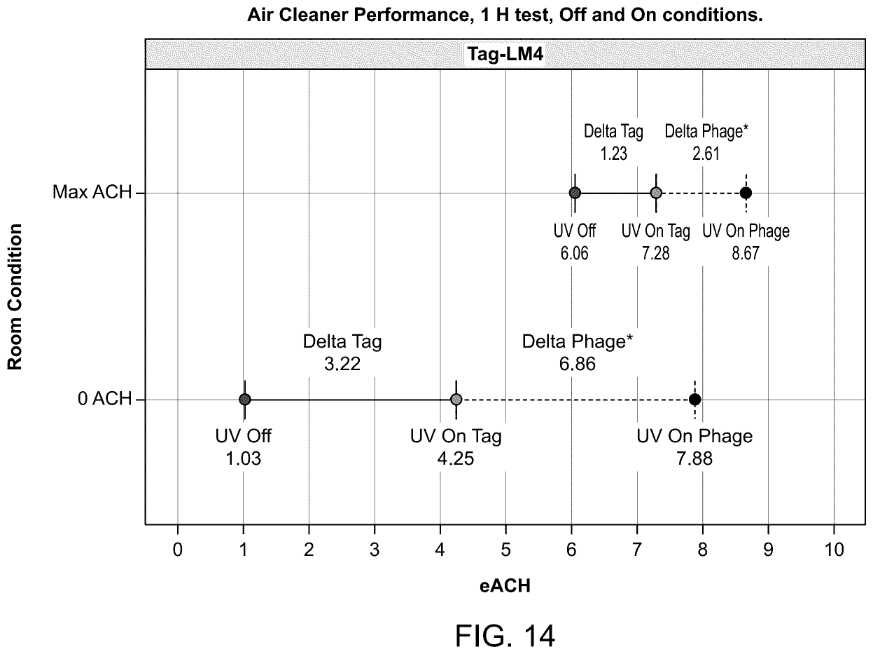

FIG. 14 illustrates an example of how using an air cleaner provides a measurable eACH. The data shown there is for two room conditions, one where there is no air change and one where the air change is at some maximum for the room. For both conditions, the eACH for is shown for three cases: (1) with the UV fixture off, (2) with the UV fixture on and exposing a tag, and (3) the presumed scaled result based on lab testing of UV effects on the application of a phage.

In the test, under the first condition (0 ACH), the reduction (scaled to eACH) with no UV fixture was measured at 1.3 eACH, which is well within measurement tolerances, and with the UV fixture on and using a tracer, was 4.25 eACH, meaning that the UV fixture can be attributed a contribution of 3.22 eACH. Where lab experiments with MS2 phage show that a scale between the tracer and the phage of around 6.86 eACH, that would indicate that even with no actual air exchange in an environment, that particular UV fixture effectively provides pathogen reductions equivalent to 7.88 air changes per hour when it is on.

Even in the case where there is actual air exchange, the UV fixture's effectiveness can be measured. As shown, the “Max ACH” condition provided 6.6 ACH. With the UV fixture on and tracer used, the measured eACH was 7.28, for an increase of 1.23 due to the UV fixture. Using experimental data showing a delta between tracer reduction and phase reduction in similar circumstances, the particular UV fixture effectively provided pathogen reductions equivalent to 8.67 air changes per hour when it is on in an environment that provided an actual 6.6 ACH.

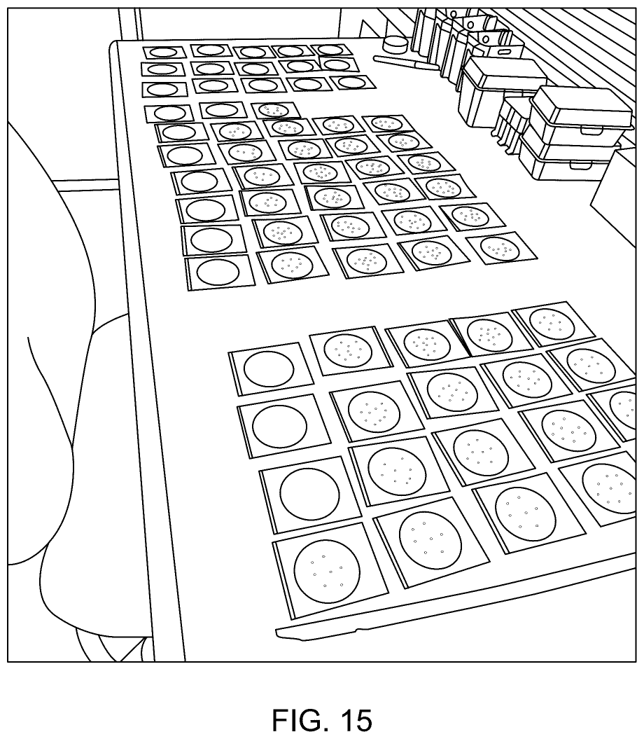

FIG. 15 illustrates an example of foil coupons having tracers deposited thereon as described elsewhere herein.

### OTHER EMBODIMENTS

Other embodiments might be implemented for introducing UV-sensitive aerosol or solid tracers for in-situ testing of an environment under test such as a building or environment that might be inhabited by humans or other organisms and might also harbor pathogens if such pathogens are introduced therein. The tracers can be used in variant forms, such as a pair of tracers or more than two tracers having different UV sensitivities (at least among two of the tracers) to provide a baseline, control, and/or reference measurement.

In some embodiments, the tracers are UV-sensitive DNA-tagged aerosol tracers that can be constructed using, for example, DNA-tagged aerosols created by SafeTraces with varying UV sensitivity. Such aerosol tracers can be tagged for later identification and can be made to mimic coughing, sneezing, etc. and have similar particle sizes, evaporation rates, etc. A tracer might include a carrier liquid, a DNA tag, and other components such that when aerosolized, they desiccate and shrink to particle sizes within the range of expelled human emissions.

Varying UV sensitivity might be provided by varying the DNA tags used in the tracer that have varying sensitivity to germicidal UV wavelengths. PCR can be used to detect DNA tags and UV damage to those DNA tags. Using measurements or pre-determined laboratory results, a testing service or tester can correlate a decay of a tracer's UV response with the UV inactivation decay of the MS2 phage and then correlate that to presumed decay of a pathogen of interest as a result of UV application by a UV fixture in the building or environment without requiring the introduction of the pathogen (or MS2) into the building or environment. A value representing that presumed decay can then be translated into an effective airflow change per hour (eACH) that the UV fixture contributed to.

Existing methods of testing effectiveness of UV fixtures in reducing concentrations of viable pathogens might involve taking radiometric measurements of intensity at an installation site and using MS2 phage inactivation studies to map intensity to phage inactivation. Another method might involve aerosol test chamber studies and particle counters, or scenario-based computational modeling. Such methods are typically undesirable and/or limiting as a tester cannot practically spray bacteriophage in healthcare and occupied spaces during testing, radiometric methods cannot model airflow in the actual environment where the UV fixture is installed, and scenario-based computational modeling requires each room to be painstakingly modelled. Furthermore, such approaches might not account for air mixing behavior and often cannot take into account environmental factors at the actual site.

Methods described herein can overcome such shortcomings as the tracers can be used and released in the actual environment under test such that they would undergo the actual airflow and air exchange that occurs in the environment and such testing can be done before or even during occupancy given that the tracers, including aerosolized tracers, can be made to be nonpathogenic, even food-safe or generally regarded as safe (GRAS).

Such testing can be conducted in-situ and can be used during installation of fixtures, during occupancy, and/or during annual, semi-annual, or other periodic testing. With the aerosolized tracers, airborne pathogen behavior can be mimicked and be safe for human exposure and used in occupied spaces. Even with low quantities of collected tracer, using PCR precise measurements of tracer quantities can be obtained. With two (or more) different sensitivities of tracers to UV exposure, ratios can be determined and effectiveness attributed to the UV fixture. The measurements can be correlated with known airborne pathogen inactivation dosages and repeated measurements can accounts for dynamic conditions in a tested space. Using aerosol-based tracers to mimic airborne pathogens and having those tracers DNA-tagged to be selectively tuned to UV sensitivity, indoor air quality and UV contributions to clean air can be quantified.

The methods described herein for measuring UV effectiveness and optimizing indoor air quality can be used in diverse settings. A chemically inert and non-hazardous system could be used, which enables UV testing in occupied spaces. Precise measurements can be made on UV-sensitive DNA-tagged tracers and their response to UVGI fixtures by employing quantitative droplet digital PCR methodology. By offering a nuanced understanding of the impact of UVGI fixtures on airborne particles and scaling the results to viral surrogates like bacteriophage MS2, this method allows a detailed understanding of UV contributions to public health and safety considerations in the built environment.

Correlation between log reductions of the DNA tracer and the MS2 bacteriophage, along with a scaling factor, can provide a quantitative method of evaluating a room's unique ventilation and pathogen disinfection effectiveness. Thus, if there is a target This aligns with recent CDC guidelines recommending a minimum of 5 air changes per hour to reduce the risk of exposure to pathogens, by providing a new technology that can be used to verify the performance of air cleaning interventions. Our approach allows for a practical assessment of whether a space meets or exceeds the recommended air changes per hour, contributing to the establishment of healthier indoor environments. These findings have broader implications for public health, emphasizing the importance of precise monitoring and understanding of complex airflow dynamics in diverse building settings.

### Study-Experimental Results

Various experiments were performed to test some of the methods and apparatus described elsewhere herein. Some of the experiments might use some of the novel and nonobvious methods and apparatus described herein but for some aspects of an experiment, less than all, or none, of the methods and apparatus might have been used. This section describes a specific study, and it should be apparent upon reading this disclosure as to how the study results can be used in implementing methods and apparatus described herein.

### A. Introduction

The study described in this section relates to evaluating a test method based on a UV-sensitive aerosol tracer where the tracer can include a DNA sequence and the tracer is such that it might have decay behavior that is similar to that of other airborne pathogens. The airborne pathogens simulated by the tracer might be from a human emission in order to address the lack of practical aerosol test methodologies or other pathogens that might have UV-sensitivity. Other nucleic acids might be used. Nucleic acids, such as DNA, are ideal model substrates because they are one of the major components of viruses and bacteria that are susceptible to damage by UV wavelengths, which contributes to their germicidal inactivation [10]. The DNA-tagged liquid released in aerosol form is quantifiable and its concentration is sensitive to deposition, filtration, dilution, and UVGI exposure. Harding introduced the use of DNA for tracking aerosol movement. One of the main advantages noted in their study was the ability to distinguish tagged particles from background aerosol concentration (given that the DNA sequences used are not naturally present in the air), and that the DNA can be carried in food additives generally recognized as safe [11]. DNA-based aerosol tracers are commonly used to assess ventilation and filtration of HVAC systems in the built environment [12].

Our study utilized a DNA tracer system comprising UV-resistant (Tag-D1) and UV-sensitive (Tag-LM4) sequences, designed to measure physical removal effects separately from UV-C radiation exposure. By employing controlled UV exposure experiments and conducting aerosolization studies in commercial buildings, we aimed to provide empirical data linking DNA-tagged aerosols to bacteriophage aerosol UV response, shedding light on the intricate dynamics of UV-sensitive aerosols indoors. This multidimensional approach contributes to the broader discussion of optimizing IAQ and safety, acknowledging the diverse settings and the need for context-specific solutions. The system, utilizing short DNA molecules as tracer molecules and analyzed via droplet digital polymerase chain reaction (ddPCR), offers a promising means of quantifying DNA-tagged aerosols on air filters and assessing their removal by ventilation, with the inclusion of a UV-sensitive tag facilitating quantification of UV-C exposure. Using these techniques, one can develop and deploy UV-sensitive aerosol technology while understanding and/or modelling interaction between airflow dynamics and UV-C fluence rate fields within environments equipped with UVGI fixtures. Differential analysis between the UV-resistant and UV-sensitive tracers allows assessment of the impact of UVGI fixtures on the local environment, aiding in estimating aerosol-associated pathogen inactivation achieved through UVGI deployment. Some of the techniques provided in this section might be used to quantify physical separation effects by filtration or air exchange, as well as pathogen inactivation resulting from UV-C exposure, and can be applied directly in occupied settings, eliminating the need for translation from test to application environments.

This study demonstrates the possibility of using selected DNA sequences as indicators of UV-C exposure by means of their decay rate in surfaces and aerosol form and to correlate DNA reduction rate due to UV-C exposure with a pathogen surrogate microorganism, such as bacteriophage MS2, in aerosol form inside a controlled test environment. Using the techniques shown herein, one can show and measure the DNA sequences' applicability in a commercial setting as a way to assess the effectiveness of different UVGI systems used for pathogen control.

### B. Materials and Methods

### 1. DNA-Tagged Liquid Suspension Preparation

The DNA-Tag tracer solutions were formulated with 99% water and 1% solids, composed of DNA and proprietary, non-toxic components that are generally regarded as safe (GRAS) by the FDA. The DNA oligonucleotides, purchased from IDT (Integrated DNA Technologies, Coralville, IA), were custom sequences of fewer than 200 base pairs each. The UV-sensitive tag sequence underwent prior optimization for its response to UV radiation emitted by an ESCO CRF UV-30A low-pressure mercury arc lamp (nearly monochromatic emission at a characteristic wavelength of 254 nm).

The UV-sensitive tracer was designed to incorporate UV-sensitive DNA sequence motifs known to produce photolesions upon UV exposure, such as “pyrimidine dimers, cyclobutane pyrimidine dimers (CPDs) and pyrimidine-pyrimidone (6-4) photoproducts [(6-4) PPs] between adjacent bases in DNA” [13,14]. Such DNA damage inhibits the replication of the UV-exposed DNA during amplification in a PCR reaction [15]. This results in a signal reduction from the UV-exposed DNA that can be measured from fluorescence signal generated in a TaqMan assay that targets the DNA molecules. The UV-resistant tag was designed to exclude thymine dimers and sequences prone to CPDs. The DNA-tags have been qualified to be stable under ambient office lighting conditions and have a shelf life of at least nine months.

Proprietary sequences of the DNA molecules used as tracers were inspired by naturally occurring sequences that would not be found in the test environment, as well as published literature on UV effects on DNA sequences [16]. The concentration of DNA in the tag solution was approximately 4,875,000,000 copies per μL for the UV-sensitive tag and 3,700,000,000 copies per μL for the UV-insensitive tag, as determined by TaqMan assays via ddPCR (QX200 system, Bio-Rad Laboratories) after extraction from coupons with an extraction buffer (see below methodology).

### 2. DNA-Tag Sensitivity to UV Radiation on Surfaces

Coupons were created by applying 50 μL of the tag solution to foil coupons (PCR plate heat seals, Bio-Rad Laboratories, Hercules, CA part number 1814040), air-drying them, and subsequently exposing them to a controlled range of UV-C doses. Rectangular foil coupons (dimensions: 6.98 cm by 8.26 cm) were used for exposure of the DNA tags to UV-C radiation. A circle (diameter=5.8 cm) was drawn on each coupon and 50 μL of tag solution was spotted and air-dried within the circle. Foil coupon UV-C exposures were conducted under collimated beams that were built around the UV-C sources described herein.

The optically-filtered KrCl lamp emits peak irradiance at 222 nm, with a full width at half maximum (FWHM) bandwidth of roughly 2 nm. The low-pressure Hg lamp is essentially monochromatic at 254 nm, with a FWHM bandwidth of less than 1 nm. The UV LEDs used in this experiment have an emission peak at about 282 nm, with a FWHM bandwidth of roughly 10 nm. All three sources have minor emission lines at longer wavelengths.

For each UV source type, exposures were controlled to achieve delivered UV doses of 10, 20, 30, 50, and 100 mJ/cm². Triplicate exposures were conducted for each dose. Exposure times required to deliver these target doses were calculated based on measured values of incident irradiance at the horizontal planar surface located below the end of each collimated beam where samples were exposed. Incident irradiance at these locations was measured for each collimated UV source using a radiometer (International Light IL-1700 with SED-008 detector), calibrated against a NIST-traceable standard at each of the peak wavelengths indicated in Table 1. Following UV exposure, coupons were returned to SafeTraces laboratories (Pleasanton, California) in light-protective bags for DNA quantification by ddPCR.

TABLE 1

| UV Lamp Type | Peak Wavelength (nm) |
|---|---|
| Optically-filtered KrCl excimer | 222 |
| Low-pressure Hg | 254 |
| UV LED | 282 |

### 3. Bacteriophage Liquid Suspension

Pure strains of the MS2 bacteriophage (ATCC 15597-B1) and its host bacterium (E. coli ATCC 15597) were obtained from ATCC by ARE Laboratory (Aerosol Research and Engineering Laboratories, Inc.) at Overland Park, Kansas City, USA. To culture MS2, liquid media was inoculated with the bacteriophage during the logarithmic growth phase of the host bacteria. After an appropriate incubation period (37° C., overnight 16 h±2 h), cells were lysed, and cellular debris separated by centrifugation. MS2 yields were greater than 10¹¹ plaque forming units (pfu) per mL with a single amplification procedure. The stock suspension was then diluted with phosphate buffered saline (PBS, Bioland Scientific LLC; Cat: PBS01-3) to a target of 10¹⁰ pfu/mL, before it was used to generate MS2-tagged aerosols with a pneumatic nebulizer (SafeTraces, Inc., model ES-1 eSprayer device).

### 4. Aerosol Generation Device

In the tests, a device worked with a pressurized air tank at 448.2 kPa, connected by a line to a liquid reservoir and a spray nozzle. The liquid reservoir contained the target tracer at a known concentration. The DNA-tagged liquid suspension and the bacteriophage liquid suspension were sprayed independently and simultaneously using the same model of pneumatic nebulizers (SafeTraces, Inc.). The particle size distribution generated by this pneumatic nebulizer using a similar DNA tracer solution was measured with a laser diffraction instrument (Spraytec, Malvern Panalytical Ltd.), placed 0.15 m from the nebulizer nozzle, the volumetric median particle size was 24.5±0.25 μm (SD from triplicate) with 10th and 90th percentiles of 6.96 and 79.23 μm, respectively [12].

### 5. Aerosolization of DNA-Tagged and Bacteriophage Suspensions in Test Chamber

To test sensitivity to UV radiation, liquid suspensions were aerosolized within a 1,050 cubic feet (30 m³) test chamber with both DNA-Tagged and MS2 bacteriophage. Four UVGI fixtures were used for UV exposure as shown in Table 2.

TABLE 2

| | | | | Distance | |
|---|---|---|---|---|---|
| | Light | Wave- | UV | from | Radiometer |
| Device | Source | length | Irradiance | Device | Used |
| Lexus 2.1 | Mercury | 254 nm | 260 μW/cm² | 1 meter | General |
| Aeromed | Lamp | | | | UV512C |
| Krypton | krypton- | 222 nm | 13 μW/cm² | 0.48 meter | Hopocolor |
| Far UV | chloride | | | | Model |
| | | | | | 220UVGI |
| | | | | | fixture |
| SUVOS-25 | LED | 275 nm | 26 μW/cm² | 1 meter | General |
| Bolb | | | | | UV512C |
| AB24 | LED | 275 nm | 1200 μW/cm² | 430 mm | International |
| AuraBlue | | | (inside) | from the | Light |
| | | | | LED inside | ILT770-UV |
| | | | | device | |

The Aeromed Lexus 2.1 device is an upper room UVGI device at 254 nm; the Far Krypton UV device is a whole room irradiation device at 222 nm wavelength [18-20] that is optically filtered from 200-230 nm; the Bolb SUVOS-25 device is an LED device for unoccupied space disinfection at 275 nm wavelength; and the AuraBlue AB24 device is an air troffer with enclosed 275 nm UV LEDs, with an internal fan that was run at 160 CFM (271.8 m³/h), low power mode, but could be run at a flow rate as high as 430.3 CFM (731 m³/h).

In these tests, the Lexus 2.1 Aeromed device is a mercury lamp at 254 nm with a UV irradiance of 260 μW/cm² at a distance of 1 meter (measured with a General UV512C radiometer), the Krypton Far UV device is a krypton-chloride (KrCl) lamp at 222 nm with a UV irradiance of 13 μW/cm² at 0.48 meter from the lamp (as measured with a Hopocolor Model 220UVGI fixture), the SUVOS-25 Bolb LED lamp at 275 nm with a UV irradiance of 26 W/cm² at 1 meter from the device (as measured with a General UV512C radiometer), and the AB24 AuraBlue LED lamp at 275 nm with an internal (inside the device) UV irradiance of 1200 W/cm² at 430 mm from the LED inside device (and no measured UV irradiance outside the device) as measured with an International Light ILT770-UV radiometer.

A control condition with no UV device was included to assess natural aerosol decay. The UV devices were placed in the chamber successively, oriented to the center of the room, and removed before the next device was tested. At least three independent replicates of aerosol release, exposure to UV from the device for 1 h, and sample collection were performed. An Optical Particle Sizer (OPS) (TSI, Inc., model 3330) was used to monitor the aerosolized particle size distribution, which was supported by monitoring the total particle concentration. Between experimental replicates, the air from the chamber was purged through HEPA filters to outdoor air between 15 to 20 minutes until particle concentrations reached background levels confirmed by the OPS in real time.

Inside the test chamber, the simultaneous aerosolization of 5 mL DNA-tagged tracer and 5 mL bacteriophage MS2 in PBS and Tryptic soy broth (Neogen, Cat: NCM0004A) were performed using two separate pneumatic nebulizers to prevent potential mixing interactions that could interfere with DNA-tag sensitivity.

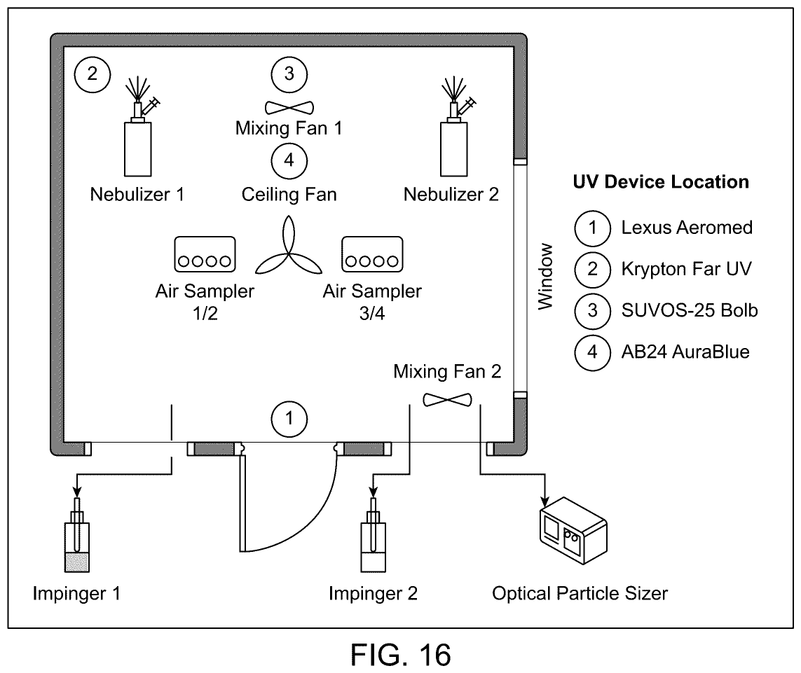

FIG. 16 illustrates an experimental chamber setup. Two mixing fans located as shown in FIG. 16 and inclined 45 degrees upward from the horizontal plane were operated during the initial five minutes post-aerosolization to promote mixing. Fan specifications included Mixing fan 1 (Amazon, model fanmk01) providing 197 CFM (334.7 m³/h), Mixing fan 2 (Honeywell, model HTF1220B) producing a flow of 478 CFM (812.1 m³/h), and one ceiling fan (4.5 ft diameter) maintaining continuous airflow at 613 CFM (1041.5 m³/h). Temperature and relative humidity were monitored throughout all experiments (AC infinity, model Controller 69), with ranges of 17.8-24.4° C. and 39%-50% RH, respectively (with a maximum change of 1.1° C. and 1% RH within replicates).

### 6. Air Sampling

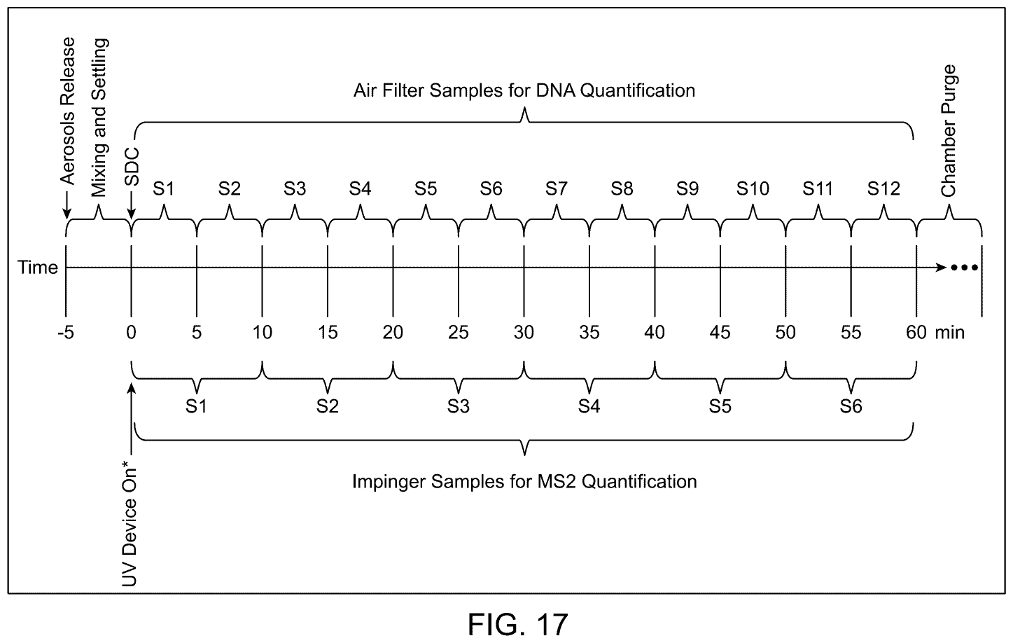

FIG. 17 illustrates an air sampling timeline, wherein “SDC” refers to a start of a decay curve and the SUVOS-25 Bolb device was turned on at t=−5 minutes. DNA-tagged aerosols were sampled with an automatic air sampling system (SafeTraces, Inc., model AS-4 AutoSampler device). The setup used four devices, each one capable of collecting four sequential air filter samples given a defined sampling interval duration, as shown in FIG. 17. The system was equipped with a pump calibrated at 5.5±0.1 L/min, and the air passed through a 25 mm glass microfiber filter with a 1.0 μm nominal pore size (grade A-E, Sterlitech Corp.). This allowed for precise and controlled sample collection, ensuring the retrieval of airborne particles containing the DNA tracer. The filters were held inside a cassette (25 mm, clear styrene, SKC PN 225-2-25LF), and at the end of the programmed sampling period collected and sent to the laboratory (SafeTraces) for DNA quantification.

Additionally, two AGI-30 impingers were used to sample the air inside of the chamber for bacteriophage quantification. These impingers were connected to two ports on opposite ends of the chamber to obtain a representative air sample. The AGI-30 impingers were designed with a critical orifice that pulled at precisely 12.5 LPM when connected to a vacuum. These impingers impacted the aerosol into 20 mL of PBS with an addition of 0.005% volume of Tween 80 (Sigma-Aldrich; Cat P4780). Each sample was collected for a period of 10 minutes to get a suspension of bacteriophage from the aerosols in the air. For each sampled interval, the two suspensions were mixed and sent to the laboratory (ARE) for quantification.

### 7. DNA Tracer Quantification

To elucidate the impact of UV exposure on the DNA tags, the highly sensitive quantitative ddPCR approach was used. This technique allowed for the precise quantification of specific DNA sequences. ddPCR utilizes water-oil emulsion technology to fractionate the reaction volume into approximately 20,000 droplets. Each droplet underwent PCR amplification, which enabled the measurement of thousands of amplification events within a single reaction well.

The DNA tags from the foil coupons were extracted for subsequent analysis by washing with a proprietary aqueous buffer solution. Similarly, the Sterlitech 1-micron (AE2500) cassette filters integrated into SafeTraces air samplers were subjected to DNA extraction using the same buffer composition used for the foil coupons. The samples were diluted to an appropriate concentration to fall within the optimum range (0 to 5000 copies per μL) for the ddPCR system. The comprehensive sample processing and dilution steps were implemented to guarantee that the subsequent ddPCR analysis would yield reliable quantitative results, enabling a detailed assessment of the impact of UV exposure on the DNA tags under various experimental conditions.

The reagents utilized in the ddPCR amplification included ddPCR Supermix for Probes No. dUTP [ddPCR Part Number: 1863025] and two TaqMan probe assays. Each TaqMan probe was unique to one sequence, enabling discrimination between the two targets by the use of distinct fluorophores. The TaqMan probe for the UV-resistant tag was labeled with the HEX fluorophore, while the TaqMan probe for the UV-sensitive tag was labeled with the FAM fluorophore. Each TaqMan probe was custom ordered and acquired from IDT.

To generate the droplets required for ddPCR, Droplet Generation Oil for Probes [ddPCR Part Number: 1863005], ddPCR 8-Well Cartridges [ddPCR Part Number: 1864008], ddPCR Gasket Seals for 8-Well Cartridges [ddPCR Part Number: 1863009], a DG8 Cartridge Holder, and a QX200™ Droplet Generator were used. The consumables used in ddPCR analysis were ddPCR 96-well Plates [ddPCR Part Number: 12001925] and PCR Plate Heat Seals [ddPCR Part Number: 1814040]. To perform the ddPCR analysis a PX1 PCR™ Plate Sealer, a C1000 Touch™ Thermal Cycler, and a QX200™ Droplet Reader were utilized.

### 8. DNA-Tracer Quantification Experimental Protocol

Each diluted DNA sample, extracted from foil coupons and air sampler cassettes, was subjected to ddPCR analysis using the specified TaqMan probes. The amplification reactions were performed on a ddPCR thermal cycler, and the resulting droplets were analyzed using the QX200 Droplet Reader. The reagents used are shown in Table 3. This method allowed for the quantification of both UV-sensitive and UV-insensitive tags, providing a robust means to assess the effectiveness of UV exposure in altering the DNA tags' integrity. The utilization of specific TaqMan probes and associated consumables ensured the accuracy and reliability of the ddPCR-based analysis. The particular thermal cycling protocol used in DNA analysis is shown in Table 4.

TABLE 3

| | Volume per | Final |
|---|---|---|
| Reagent | Reaction | Concentration |
| ddPCR Supermix for Probes | 12.5 μL | 1X |
| no dUTP, 2x | | |
| UV-Sensitive Primer/Probe, | 2.5 μL | 1X, 500 nM Primers, |
| FAM, 10X | | 250 nM Probe |
| UV-Insensitive Primer/Probe, | 2.5 μL | 1X, 500 nM Primers, |
| HEX, 10X | | 250 nM Probe |
| Nuclease-Free Water | 3.5 μL | N/A |
| DNA Sample | 4 μL | Sample Dependent |

TABLE 4

| Step | Temperature | Time | | Cycle |
|---|---|---|---|---|
| Polymerase activation | 95° C. | 10 | minutes | 1 |
| Denaturation | 94° C. | 30 | seconds | 39 |
| Annealing/Extension | 60° C. | 2 | minutes | |
| Signal Stabilization | 98° C. | 5 | minutes | 1 |

| | | | |
|---|---|---|---|
| Hold | 12° C. | ∞ | N/A |

### 9. Bacteriophage Quantification

The liquid suspensions obtained from impingers sampling air from the chamber were serially diluted with PBS (containing Tween 80 as described in the section air sampling) and plated in triplicate. This was done using a standard drop plaque assay technique onto tryptic soy agar plates (Hardy Diagnostics) with its host (E. coli). Plates were incubated at 37° C. overnight (between 12 to 16 hours) and the enumeration was recorded by quantifying the plaques formed on the plates [22]. Technical triplicate results from each sample were averaged, and the concentration in the sample suspension as pfu/mL was determined. This value was used to calculate the total pfu collected in the sample (based on the 20 mL collected), with this and the total volume of air sampled the calculation of the concentration as pfu per L of air was obtained.

### 10. Statistical Analysis

Data organization, data cleaning, figure generation, and statistical analysis as illustrated herein can be performed with R version 4.0.3 and packages contained in the Tidyverse [23,24] software. For coupons, the total DNA copies quantified per sample were transformed to Log base 10, and this value was used as the dependent variable in a linear regression model with dose as the independent variable. For liquid aerosols containing DNA-tags or MS2 bacteriophage, quantified results as concentration in units of DNA copies per L of air, or pfu per L of air were used to calculate their decay rate, which was assumed to be a first-order process. Within experimental replicates, each value was standardized by calculating the natural log of the ratio of concentration after a given exposure period to the initial concentration observed in the same experimental replicate of aerosol emission and sampling as shown in Equation 1, wherein “Aerosol Tag Reduction” is a normalized natural log reduction of the DNA-Tag or MS2 bacteriophage concentration, Cᵢ is a concentration at time t=i, and C₀ is an initial concentration as a reference point, defined as the time zero minutes from aerosols decay.

$$Aerosol Tag Reduction = Ln ( {C}_{i} / {C}_{0} ) \qquad ( Eqn . 1 )$$

The aerosol tag reduction values were transformed to positive magnitude and used as the dependent variable in a regression model using time as the independent variable. The initial time (t=0) was defined as five minutes after aerosols release to provide enough time for aerosols to mix in the air and a stable log-linear decay rate measurement. This point is referred to herein as the “Start of the Decay Curve” (SDC), similar to how it is described in the ASHRAE standard 241 Appendix C [26]. An exception was made for the SUVOS-25 Bolb device, for which the SDC was defined to be ten minutes after aerosols were released, given that when observing the data, it was noted that more time was needed to have a log-linear decay for this device setting for the bacteriophage MS2. This exception was necessary, because the SUVOS-25 Bolb device had to be on during the initial air mixing period of five minutes with mixing fans on, albeit it was rigged with a flap to cover the UV LEDs, which was triggered to drop after the five minutes. This was not the case for the other three devices tested, which were turned on remotely after five minutes of air mixing. We believe that even though the flap was to prevent early exposure to UV from SUVOS-25, there was some aerosol being affected by UV behind the flap in the aerosol mixing period, which may have contributed to some signal instability during that time. The SUVOS-25 Bolb device is not intended for occupied spaces. Air mixing is important for reaching equilibrium of the aerosol and is allowed in testing according to the ASHRAE 241 standard. Other time periods might be used instead.

One model slope was obtained for each aerosol tag (MS2, Tag-D1, Tag-LM4) and device condition (UVGI off as control, plus four devices) combination (total=15). The slope of the decay rate was interpreted as the equivalent air changes per hour (eACH) using the expression in Equation 2, similar to how air changes per hour are calculated in the ASTM Method E741-11, which is used for measuring the air change rate in a room with outdoor air by means of a tracer gas [27]. In Equation 2, “Y” is an additive inverse of the Aerosol Tag Reduction (as might be computed per Equation 1), “a” is a slope, interpreted as eACH, “X” is a time elapsed since Start of Decay Curve, in hours, and “b” is the Y value when time equals zero (intercept).

$$Y = a X + b \qquad ( Eqn . 2 )$$

For all the models, a significance level of 5% was used to reject the null hypothesis of no reduction given increased time (or dose) of exposure to UV.

The effectiveness of each UVGI device was also calculated in the form of equivalent clean airflow delivery rate as described in the ASHRAE standard 241 Appendix A for getting a better understanding on scalability of the results and as illustrated by Equation 3, wherein VACS is an air cleaning system equivalent clean airflow rate, in CFM, V is a test chamber volume, in cubic feet (ft³), K_{td} is an infectious microorganism (MS2) decay rate with an air cleaning system operating, in minute⁻¹ units, and K_{nd} is an infectious microorganism (MS2) decay rate without air cleaning system operating, also in minute-1 units.

$${V}_{ACS} = V * ( {K}_{td} - {K}_{ad} ) \qquad ( Eqn . 2 )$$

### C. Results

### 1. UV Sensitivity on Surfaces

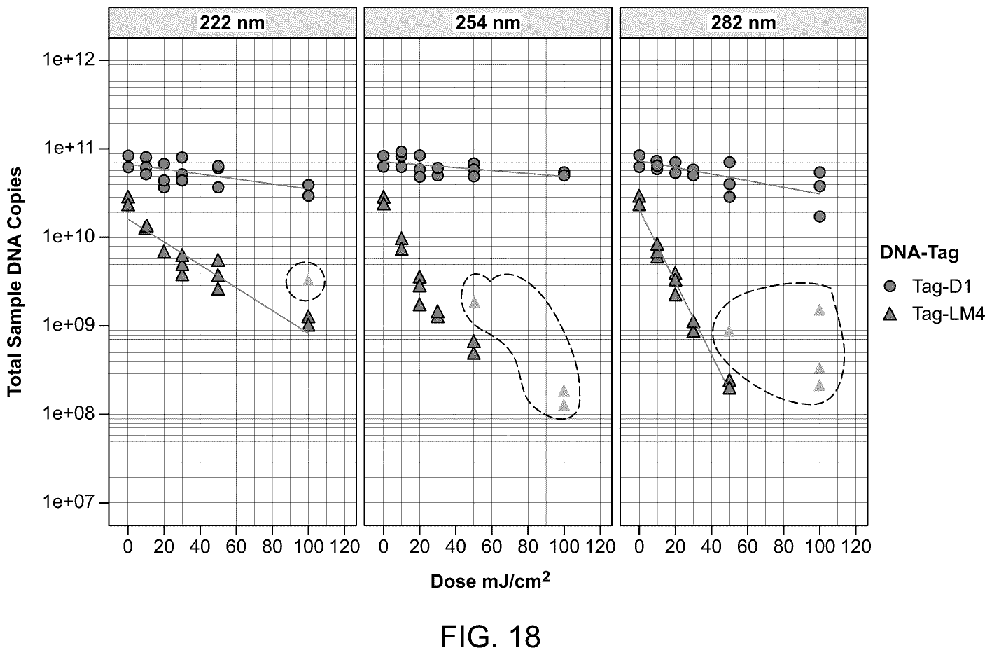

FIG. 18 provides plots of results of UV exposure experiments on foil coupons, focusing on the UV-sensitive and resistant tags. The investigation covered UV wavelengths of 222 nm, 254 nm, and 282 nm, with doses ranging from 0 to 100 mJ/cm². The y-axis in each of the three plots represents the total DNA copies measured using ddPCR, while the x-axis plots the dose in mJ/cm². Values for the UV-resistant Tag-D1 are shown by circular data points and the values for the UV-sensitive Tag-LM4 are shown by triangular data points. As shown, some of the triangular data points (enclosed in dashed curves) are excluded from curve fitting.

Table 5 summarizes results used to create a linear model of DNA-tag decay based on the dose applied on coupon surfaces, showing foil coupon DNA copies data ranges used for estimation of relationship between UV-dose and DNA-Tag change. Doses of UV at the three wavelengths tested ranged from 0 to 100 mJ/cm² for DNA-tagged tracer D1 and 0 to 50 mJ/cm² for DNA-tagged tracer LM4 in the linear range of response for tracer decay as shown in FIG. 18. A total of 14-18 observations (N) were made for the data sets in Table 5. The range of copies of DNA quantified from the extraction of the coupons is reported as minimum and maximum values in Table 5.

TABLE 5

| | | | Dose Range | | | |
|---|---|---|---|---|---|---|
| | Wavelength | | (in mJ/cm²) | | Range of DNA copies per Sample | |
| DNA-Tag | (in nm) | N | Min | Max | Min | Max |
| Tag-D1 | 222 | 18 | 0 | 100 | 30,031,250,000 | 83,437,500,000 |
| | 254 | 18 | 0 | 100 | 47,625,000,000 | 91,562,500,000 |
| | 282 | 18 | 0 | 100 | 16,875,000,000 | 83,437,500,000 |
| Tag-LM4 | 222 | 17 | 0 | 100 | 971,875,000 | 27,406,250,000 |
| | 254 | 14 | 0 | 50 | 468,750,000 | 27,406,250,000 |
| | 282 | 14 | 0 | 50 | 187,500,000 | 27,406,250,000 |

Distinct patterns were revealed in the UV dose response of the UV-sensitive tag (Tag-LM4, triangles on FIG. 18) at each wavelength. At 222 nm, a linear dose response was observed over the entire range of doses up to 100 mJ/cm², showing a reduction in DNA copies with increasing UV dose. For 254 nm and 282 nm, the UV-sensitive tag demonstrated a linear response up to 50 mJ/cm², beyond which the reduction in signal became non-linear (represented by the triangular data points in dashed line regions, removed for statistical analysis). For Tag-LM4 using 222 nm, one observation at 100 mJ/cm² was noted to be highly influential with a Cooks' distance of 1.24. It was therefore removed from the analysis before model estimations [25,28]. Separately, in the case of Tag-LM4 and the wavelength 254 nm and 282 nm, the group of values removed was because their presence showed a non-linear trend (lack of fit due to no random errors) in the residuals values when plotted against dose as the independent variable, or subsequently showed high influence in the decay rate estimations. For Tag-LM4 and the wavelengths 222 nm, 254 nm, 282 nm, the coefficient of determination (r²) was improved to 88%, 92%, and 98% respectively from 80%, 87%, and 59% when the points that did not fit well with the log-linear models were removed. Notably, the UV-resistant tag exhibited substantially less reduction in signal across all UV wavelengths and doses (circular dots and lines, Tag-D1), indicating its resistance to the effects of UV exposure. The foil coupons exhibit large changes with the dose exposures, and it should be noted that the coupons were hand spotted and air-dried, which can contribute to some noise in the data.

Table 6 shows how decay rates might be a function of UV irradiance at wavelengths 222 nm, 254 nm, and 282 nm with respect to UV-resistant Tag-D1 and UV-sensitive Tag-LM4 dried on foil coupons. Table 6 includes exponential rates of decay, obtained from simple linear regression of the log-transformed variable DNA copies. Decay rates were estimated between 0 and 50 mJ/cm² for Tag-LM4, and between 0 and 100 mJ/cm² for Tag-D1.

TABLE 6

| | Wave- | Estimate Log₁₀ | | | | 95% C.I. Log₁₀ DNA | |
|---|---|---|---|---|---|---|---|
| | length | DNA Copies | Std. | | | Copies (in mJ/cm²) | |
| DNA-Tag | (in nm) | (in mJ/cm²) | Error | Statistic | p-value | Low | High |
| Tag-D1 | 222 | −0.0027 | 0.0008 | −3.54 | 0.003 | −0.0043 | −0.0011 |
| | 254 | −0.0016 | 0.0006 | −2.90 | 0.10 | −0.0028 | −0.0004 |
| | 282 | −0.0036 | 0.0009 | −4.8 | 0.001 | −0.0055 | −0.0017 |
| Tag-LM4 | 222 | −0.0128 | 0.0012 | −10.38 | <0.001 | −0.0154 | −0.0102 |
| | 254 | −0.0342 | 0.0028 | −12.6 | <0.001 | −0.0403 | −0.0280 |
| | 282 | −0.0416 | 0.0017 | −24.10 | <0.001 | −0.0453 | −0.0378 |

Table 7 provides data for a UV Dose (Fluence) (mJ/cm²) per 1 Log₁₀ reduction of indicator/DNA-Tagged tracer. The UV dose (mJ/cm²) per 1 Log₁₀ reduction of MS2 (Phage) ATCC 15597 is reported in the literature around 19 to 20 mJ/cm², as shown in Table 7. DNA-tag LM4 is close to the reported bacteriophage MS2 with a dose ranging between 24.0-78.1 mJ/cm² for 1 Log₁₀ reduction in the range of wavelengths tested. Tag-D1 requires at least one order of magnitude of more dose to be equivalent in response to the bacteriophage MS2, which is why it is considered more resistant to UV than DNA-tag Tag-LM4. The MS2 value in Table 7 is taken from [29]. These findings underscore the differential impact, detected by the DNA-tagged tracers, of UV exposure and UV-induced DNA damage.

TABLE 7

| | UV Dose (Fluence) (in mJ/cm²) for 1 Log₁₀ | | |
|---|---|---|---|
| Indicator/ | Reduction at Different Wavelengths | | |
| DNA-Tag | 222 nm | 254 nm | 282 nm |
| Tag-D1 | 370.4 | 625.0 | 277.8 |
| Tag-LM4 | 78.1 | 29.2 | 24.0 |
| Bacteriophage | — | 19 to 20 | — |

The observed reduction in DNA copies on a UV dose basis highlights the efficacy of UV in diminishing the amplifiable DNA content, establishing a relationship between DNA UV-induced damage assessment and its potential application in UV-exposed environments.

### 2. UV Sensitivity of DNA-Tagged Aerosols and MS2 Bacteriophage

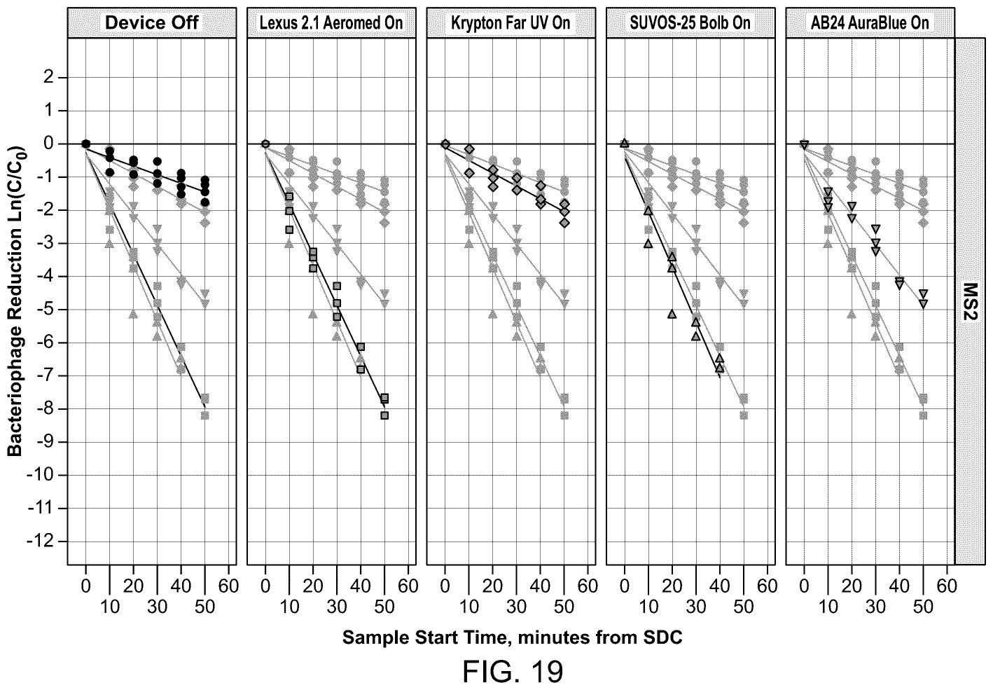

The bacteriophage MS2 sensitivity to UVGI showed a great range of variation, with reduction differences of orders of magnitude among devices. A summary of the range of values observed is presented in Table 8. The highest reduction in concentration for an individual sample was observed in the Lexus 2.1 device as measured in eACH (Table 8). However, in terms of rate (Table 9), the SUVOS-25 device showed a higher sensitivity than the Lexus 2.1, with estimated values of 9.97 eACH and 9.27 eACH respectively. The AB24 and Krypton devices followed with decay rates equivalent to 5.48 eACH and 2.30 eACH respectively. When the UVGI devices were used, all estimated decay rates were on average higher than the natural decay observed in the chamber, which was estimated at 1.53 eACH. FIG. 19 illustrates this.

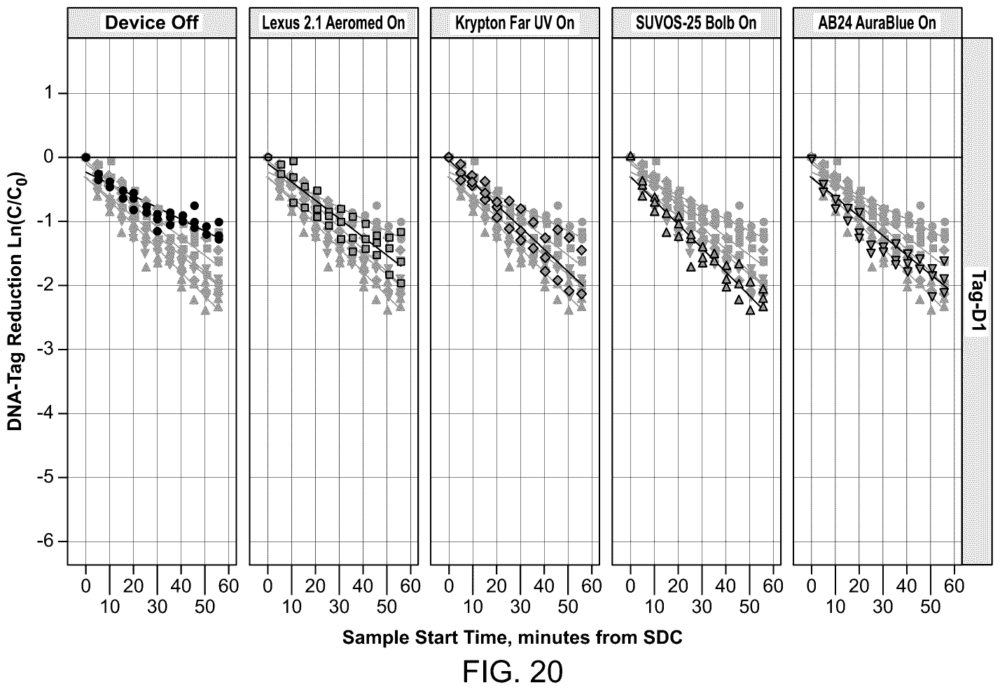

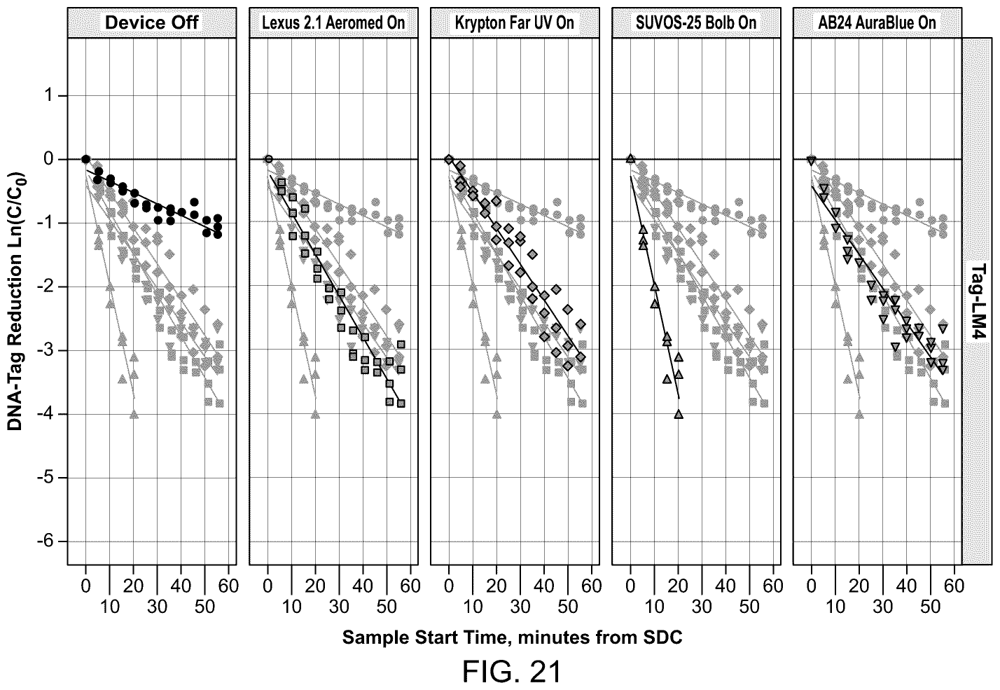

For the DNA-tagged aerosol tracers, the results of UV inactivation are shown in FIGS. 20 and 21. The maximum observed reduction for an individual sample was 2.40 eACH and 4.2 eACH for Tag-D1 and Tag-LM4 respectively (Table 10), both happening for exposure to the SUVOS-25 device. There was a clear increase in the estimated eACH given the presence of the UVGI devices (Table 11), and as expected, Tag-LM4 showed higher decay rate with more sensitivity than Tag-D1. For the SUVOS-25 device, the results followed a log-linear trend for 25 minutes. After this period, the values did not follow the same rate of reduction and were therefore omitted from the analysis.

Table 8 illustrates MS2 Bacteriophage decay in chamber summary, testing at 0 ACH with UVGI fixtures.

TABLE 8

| | | | | Decay | Phage Reduction | |
|---|---|---|---|---|---|---|
| Aerosol | UVGI Device | | | Duration | Ln(C/C₀)*(−1) | |
| Tag | Status | N | Replicates | (minutes) | Min. | Max. |
| MS2 | Device Off | 24 | 4 | 60 | 0 | 1.77 |
| Bacteriophage | Lexus Aeromed On | 18 | 3 | 60 | 0 | 8.19 |
| | Krypton Far UV On | 18 | 3 | 60 | 0 | 2.38 |
| | SUVOS-25 Bolb On | 15* | 3 | 50* | 0 | 6.77 |
| | AB24 AuraBlue On | 18 | 3 | 60 | 0 | 4.83 |

In Table 8, “*” indicates data for the Start of Decay Curve for model estimation set at 15 minutes after aerosols spray. Table 9 provides values for MS2 Bacteriophage decay in chamber models as eACH.

TABLE 9

| | | | | | | 95% C.I. | | |
|---|---|---|---|---|---|---|---|---|
| Aerosol | UVGI Device | Est. | Std. | | | Low | High | V_{ACS} |
| Tag | Status | eACH | Error | Statistic | p-value | eACH | eACH | CFM |
| MS2 | Device Off | 1.53 | 0.18 | 8.38 | <0.001 | 1.15 | 1.90 | — |
| Bacteriophage | Lexus Aeromed On | 9.27 | 0.31 | 30.9 | <0.001 | 8.62 | 9.92 | 134.85 |
| | Krypton Far UV On | 2.30 | 0.21 | 10.78 | <0.001 | 1.85 | 2.75 | 13.48 |
| | SUVOS-25 Bolb On | 9.97 | 0.64 | 15.62 | <0.001 | 8.59 | 11.35 | 146.98 |
| | AB24 AuraBlue On | 5.48 | 0.27 | 20.34 | <0.001 | 4.91 | 6.5 | 68.82 |

FIG. 19 illustrates UV Inactivation curves in logarithmic scale for aerosolized MS2 bacteriophage in a 30 m³ sealed chamber. Solid colors represent the UV device condition values inside each facet, gray shaded values inside each facet represent the rest of the data for relative comparison. Table 10 provides data for DNA Tracer decay in chamber summary, testing at 0 ACH with UVGI fixtures.

In Table 10, “*” indicates cases where a log-linear relationship for model was observed only up to 25 minutes after the start of aerosols decay. Table 11 provides data for DNA Tracer decay in chamber modeled as eACH.

TABLE 10

| | | | | Decay | DNA-Tag Reduction | |
|---|---|---|---|---|---|---|
| Aerosol | UVGI Device | | | Duration | Ln(C/C₀) *(−1) | |
| Tag | Status | N | Replicates | (minutes) | Min. | Max. |
| Tag-D1 | Device Off | 36 | 3 | 60 | 0 | 1.28 |
| | Lexus Aeromed On | 35 | 3 | 60 | 0 | 1.97 |
| | Krypton Far UV On | 36 | 3 | 60 | 0 | 2.16 |
| | SUVOS-25 Bolb On | 36 | 3 | 60 | 0 | 2.40 |
| | AB24 AuraBlue On | 36 | 3 | 60 | 0 | 2.16 |
| Tag-LM4 | Device Off | 36 | 3 | 60 | 0 | 1.19 |
| | Lexus Aeromed On | 36 | 3 | 60 | 0 | 3.85 |
| | Krypton Far UV On | 36 | 3 | 60 | 0 | 3.25 |
| | SUVOS-25 Bolb On | 15* | 3 | 25* | 0 | 4.2 |
| | AB24 AuraBlue On | 36 | 3 | 60 | 0 | 3.29 |

TABLE 11

| | | | | | | 95% C.I. | |
|---|---|---|---|---|---|---|---|
| Aerosol | UVGI Device | Estimate | Std. | | | Low | High |
| Tag | Status | eACH | Error | Statistic | p-value | eACH | eACH |
| Tag-D1 | Device Off | 1.14 | 0.8 | 14.3 | <0.001 | 0.98 | 1.31 |
| | Lexus Aeromed On | 1.74 | 0.13 | 13.90 | <0.001 | 1.49 | 2.00 |
| | Krypton Far UV On | 2.13 | 0.13 | 15.93 | <0.001 | 1.86 | 2.41 |
| | SUVOS-25 Bolb On | 2.26 | 0.11 | 19.69 | <0.001 | 2.2 | 2.49 |
| | AB24 AuraBlue On | 1.90 | 0.12 | 16.31 | <0.001 | 1.67 | 2.14 |
| Tag-LM4 | Device Off | 1.4 | 0.7 | 14.44 | <0.001 | 0.90 | 1.19 |
| | Lexus Aeromed On | 3.99 | 0.19 | 21.32 | <0.001 | 3.61 | 4.37 |
| | Krypton Far UV On | 3.39 | 0.16 | 20.92 | <0.001 | 3.6 | 3.72 |
| | SUVOS-25 Bolb On | 10.57* | 0.71 | 14.93 | <0.001 | 9.4 | 12.10 |
| | AB24 AuraBlue On | 3.25 | 0.16 | 20.47 | <0.001 | 2.93 | 3.57 |

In Table 11, the cases noted with “*” indicate that the log-linear relationship for the model was observed only up to 25 minutes after the start of aerosols decay.

FIG. 20 illustrates UV Inactivation curves in logarithmic scale for aerosolized DNA Tag-D1 in a 30 m³ sealed chamber.

FIG. 21 illustrates UV Inactivation curves in logarithmic scale for aerosolized DNA Tag-LM4 in a 30 m³ sealed chamber.

### 3. Scaling Factor

Table 12 shows the results of scaling factors for DNA-tracer decay to bacteriophage MS2 decay using linear regression, which provides estimates of eACH for each DNA-tag and the MS2 bacteriophage a relationship between their decay magnitudes was calculated as shown in Table 12. The relationship shown in Table 12 can be used to estimate bacteriophage reduction when only the DNA-tag decay rate is measured in a space.

TABLE 12

| | | Device effect | | Decay |
|---|---|---|---|---|
| | | Δ = eACH_{UV}_On − | | Relationship |
| Aerosol | | eACH_{UV}_Off | | ΔMS2/ |
| Tag | UVGI Device | ΔMS2 | ΔDNA-Tag | ΔDNA-Tag |
| Tag-D1 | Lexus Aeromed On | 7.74 | 0.60 | 12.91 |
| | Krypton Far UV On | 0.77 | 0.99 | 0.78 |
| | SUVOS-25 Bolb On | 8.44 | 1.11 | 7.59 |
| | AB24 AuraBlue On | 3.95 | 0.76 | 5.20 |
| Tag-LM4 | Lexus Aeromed On | 7.74 | 2.94 | 2.63 |
| | Krypton Far UV On | 0.77 | 2.35 | 0.33 |
| | SUVOS-25 Bolb On | 8.44 | 9.53 | 0.89 |
| | AB24 AuraBlue On | 3.95 | 2.21 | 1.79 |

### 4. Commercial Building Application of DNA-Tracer Tag

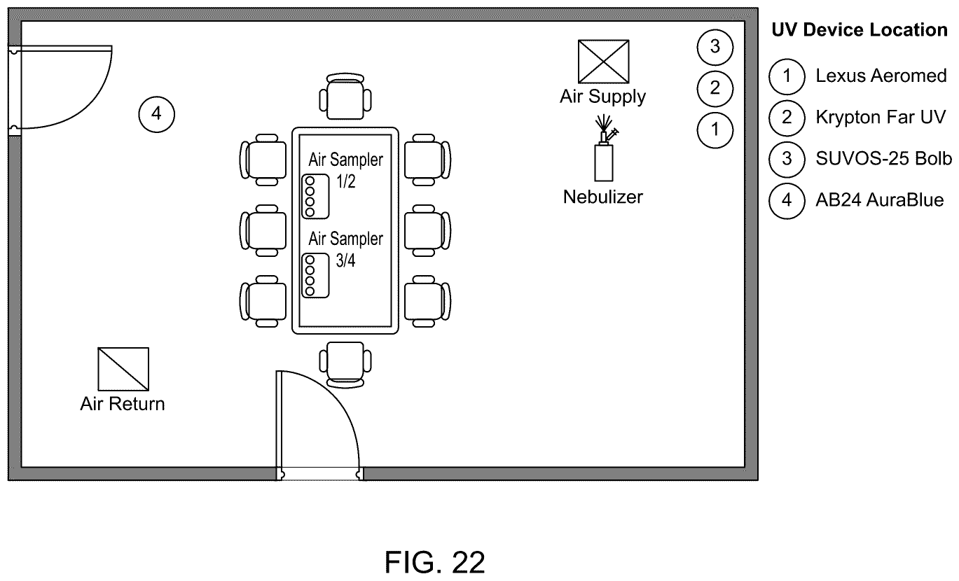

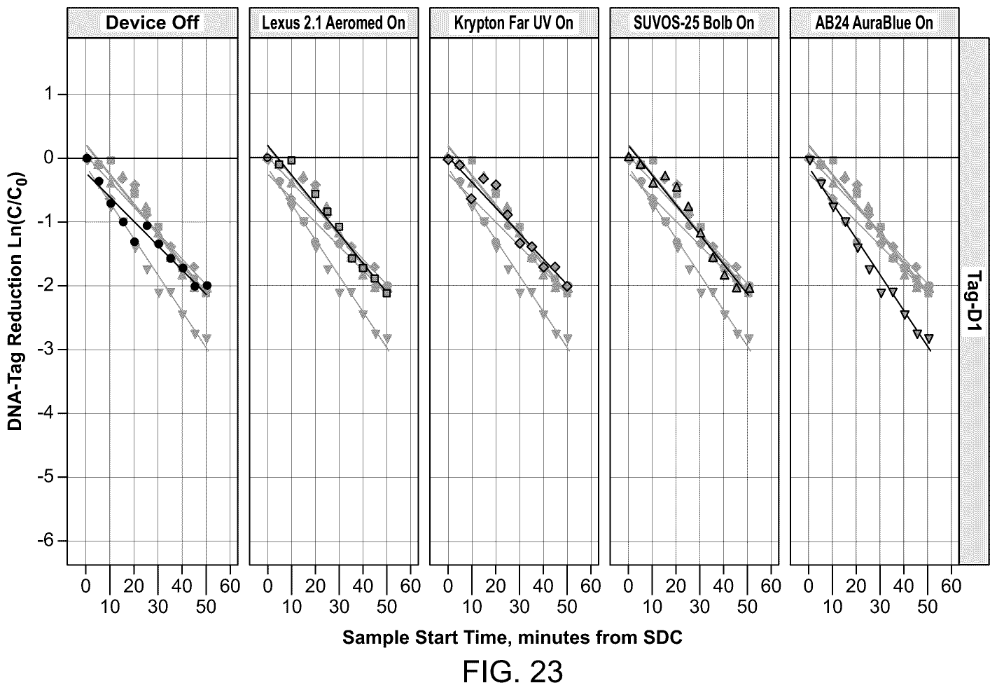

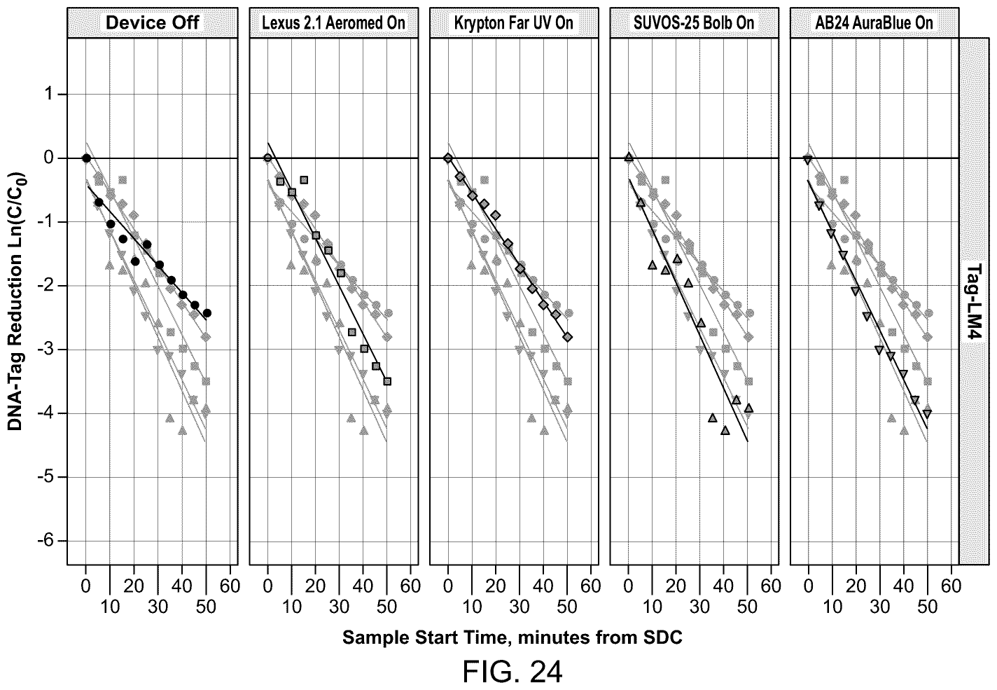

FIG. 22 illustrates a model of a conference room used to assess the sensitivity of the DNA-tagged tracers under different UVGI fixture conditions in a commercial building setting. The dimensions of the room were 4.87 m by 4.57 m and floor-to-ceiling height 2.74 m, resulting in a volume of 61.16 m³ (2160 ft³). A square ceiling diffuser supplies roughly 42 L/s of air to the space, which is exhausted via a ceiling return grill diagonally opposite the supply, as shown in FIG. 22. The supply air flow rate corresponds to an air change rate of approximately 2.5 ACH. The return vented directly into the ceiling and a portion of the air likely was picked up again at some point in the system for recirculation, the exact amount is unknown. The baseline condition, with UVGI fixtures off, provided a reference point for comparison and to determine natural loss. Subsequent experiments involved testing each UVGI fixture individually, with the tracer aerosolized in the room. The results, depicted in FIGS. 23 and 24, illustrate the tracer's responsiveness to the diverse UVGI devices employed in the study. The varying levels of sensitivity observed in response to individual UVGI fixtures highlight the nuanced interactions between the tracer and different UV technologies, offering insights into the performance of these fixtures in commercial building indoor environments and the resulting data is presented in Tables 13 and 14.

FIG. 22 illustrates UVGI configuration at a commercial conference room for effectiveness evaluation. As shown there, a room size is approximately 61.16 m³ (2160 ft³) in volume, doors closed during testing period, and the pneumatic nebulizer placed directly under the HVAC air supply. Air samplers were placed on the conference room table. The UVGI fixtures were located near the corner of the room at a 45-degree angle from the wall shown as the uppermost wall in FIG. 22 and were projecting UV towards the center of the room at various heights.

Table 13 shows a summary of result ranges from the testing at the commercial office.

TABLE 13

| | | | | Decay | DNA-Tag Reduction | |
|---|---|---|---|---|---|---|
| Aerosol | UVGI Device | | | Duration | Ln(C/C₀) *(−1) | |
| Tag | Status | N | Replicates | (minutes) | Min. | Max. |
| Tag-D1 | Device Off | 11 | 1 | 55 | 0 | 2.1 |
| | Lexus Aeromed On | 10* | 1 | 55 | 0 | 2.12 |
| | Krypton Far UV On | 11 | 1 | 55 | 0 | 2.00 |
| | SUVOS-25 Bolb On | 11 | 1 | 55 | 0 | 2.6 |
| | AB24 AuraBlue On | 11 | 1 | 55 | 0 | 2.82 |
| Tag-LM4 | Device Off | 11 | 1 | 55 | 0 | 2.43 |
| | Lexus Aeromed On | 11 | 1 | 55 | 0 | 3.50 |
| | Krypton Far UV On | 11 | 1 | 55 | 0 | 2.81 |
| | SUVOS-25 Bolb On | 11 | 1 | 55 | 0 | 4.29 |
| | AB24 AuraBlue On | 11 | 1 | 55 | 0 | 4.1 |

In Table 12, the case marked with “*” is a case where the sample size is lower due to the elimination of one outlier value.

FIG. 23 illustrates UV Inactivation curves in logarithmic scale for aerosolized DNA Tag-D1 in a 61.16 m³ conference room in a commercial building.

FIG. 24 illustrates UV Inactivation curves in logarithmic scale for aerosolized DNA Tag-LM4 in a 61.16 m³ conference room in a commercial building.

Table 14 provides a summary of EACH for UVGI devices in DNA-tracer response to exposure in a sample commercial office.

TABLE 14

| | | | | | | 95% C.I. | |
|---|---|---|---|---|---|---|---|
| Aerosol | UVGI Device | Estimate | Std. | | | Low | High |
| Tag | Status | eACH | Error | Statistic | p-value | eACH | eACH |
| Tag-D1 | Device Off | 2.27 | 0.19 | 11.96 | <0.001 | 1.84 | 2.70 |
| | Lexus Aeromed On | 2.78 | 0.17 | 16.25 | <0.001 | 2.39 | 3.18 |
| | Krypton Far UV On | 2.47 | 0.21 | 11.75 | <0.001 | 1.99 | 2.94 |
| | SUVOS-25 Bolb On | 2.79 | 0.21 | 13.39 | <0.001 | 2.32 | 3.26 |
| | AB24 AuraBlue On | 3.44 | 0.15 | 22.61 | <0.001 | 3.10 | 3.79 |
| Tag-LM4 | Device Off | 2.54 | 0.25 | 10.30 | <0.001 | 1.98 | 3.9 |
| | Lexus Aeromed On | 4.56 | 0.31 | 14.66 | <0.001 | 3.86 | 5.27 |
| | Krypton Far UV On | 3.41 | 0.11 | 31.53 | <0.001 | 3.17 | 3.66 |
| | SUVOS-25 Bolb On | 4.95 | 0.58 | 8.55 | <0.001 | 3.64 | 6.27 |
| | AB24 AuraBlue On | 4.69 | 0.23 | 20.78 | <0.001 | 4.18 | 5.20 |

Table 15 shows data from results of scaling of the commercial building results for the MS2 bacteriophage.

TABLE 15

| | | | | | Decay | | | |
|---|---|---|---|---|---|---|---|---|
| | | | | | Relationship | | | Total |
| Aerosol | | UV | UV | Delta-Tag | ΔMS2/ | ΔMS2 | V_{ACS} | MS2 |
| Tag | UVGI Device | Off | On | UV_{On-Off} | ΔDNA-Tag | Estimation | CFM | eACH |
| Tag-D1 | Lexus Aeromed On | 2.27 | 2.78 | 0.52 | 12.91 | 6.65 | 239.40 | 8.92 |
| | Krypton Far UV On | 2.27 | 2.47 | 0.20 | 0.78 | 0.16 | 5.76 | 2.43 |
| | SUVOS-25 Bolb On | 2.27 | 2.79 | 0.52 | 7.59 | 3.95 | 142.20 | 6.22 |
| | AB24 AuraBlue On | 2.27 | 3.44 | 1.17 | 5.20 | 6.10 | 219.60 | 8.37 |
| Tag-LM4 | Lexus Aeromed On | 2.54 | 4.56 | 2.3 | 2.63 | 5.33 | 191.88 | 7.87 |
| | Krypton Far UV On | 2.54 | 3.41 | 0.88 | 0.33 | 0.29 | 10.44 | 2.83 |
| | SUVOS-25 Bolb On | 2.54 | 4.95 | 2.42 | 0.89 | 2.14 | 77.4 | 4.68 |
| | AB24 AuraBlue On | 2.54 | 4.69 | 2.15 | 1.79 | 3.85 | 138.60 | 6.38 |

In Table 15, the VAcs is calculated based on a room volume of 61.16 m³ (2160 ft³) and that value represents an air cleaning system equivalent clean airflow rate.

### D. Discussion

As the disclosure here and the study data illustrates, using DNA-tagged aerosol tracers for in situ testing in commercial building settings, allowing for the differentiation of UVGI fixture contributions into air disinfection, is feasible and can be done as described herein. The tracers, exemplified by the UV-sensitive LM4, proved effective in occupied spaces and could scale with known airborne viral pathogen surrogates like MS2 bacteriophage. Other tracers might be used.

In a particular implementation, a germicidal verification system might include a dispersing unit such as one or more of those described herein can disperse a tag composition. The composition might comprise two or more tags, each comprising a nonpathogenic portion having a particular range of sensitivity to exposure to ultraviolet (UV) radiation, wherein not all tags of the two or more tags have the same range of sensitivity. These tags might be DNA-tagged aerosol tracers such as DNA-tagged aerosol tracers described herein. Examples might be the tracer Tag-D1 and the tracer Tag-LM4 described herein.

The germicidal verification system might include a conduit for providing dispersed tags in a tag composition, for being dispersed into an environment. Once dispersed into an environment under test, such as a room or enclosed space, a collection unit of the germicidal verification system might collect tag compositions that comprise some portion or portions of the dispersed tags that were dispersed into the environment after at least some of the tag compositions were exposed to some UV radiation in the environment, such as by a UVGI system or device, prior to being collected by the collection unit.

The germicidal verification system might include a measurement unit that measures amounts of the two or more tags in the collected tag composition, wherein measurements of the measurement unit of a given tag depend, at least in part, on an amount of sensitivity to UV exposure of the given tag. From the measurements, effective rates of air exchange and/or pathogen risks might be computed.

The implementation of a DNA-tracer-based approach, as demonstrated herein, offers a tool for evaluating and optimizing indoor air quality in commercial building settings in part by providing a way to measure liquid aerosols reduction by ventilation, and UVGI contributions in units of equivalent ventilation (as VACs) as shown in Table 15. The UV-sensitive tracers, when aerosolized in conjunction with the MS2 phage under controlled test chamber conditions, provide an understanding of the dynamics of these surrogate airborne particles under ideal test conditions, such as the case of zero air changes per hour, a well-mixed chamber mixed using mixing fans, and allowing time for the aerosols to reach homogeneity. Using the teachings herein, log reductions between the bacteriophage and the DNA-tagged tracers when exposed to UVGI fixture wavelengths can be scaled. This scaling permits an assessment of the contributions of UVGI fixtures to the sampling points under study and correlates the DNA-tagged tracer data to MS2 bacteriophage UV inactivation behavior.

The controlled test chamber experiments under ideal conditions demonstrated high reproducibility, and there is a log linear trend of bacteriophage MS2 and DNA-tagged tracer decay rates. This was true for the UV off conditions as well as the UV on. In the UV off condition, the MS2 bacteriophage and the DNA-tagged tracers' decay rates in the test chamber are similar, which is attributable to the matching of the chemical properties of the solutions for the bacteriophage and the DNA-tagged tracer. This allowed for similar particle sizes generated from the pneumatic nebulizers. The composition of the DNA-tagged tracers has been developed to mimic the chemical composition of human upper respiratory excretions. The same pneumatic nebulizers were used in the commercial conference room testing as the controlled test chamber, and thus provide confidence in correlating the results from the DNA-tagged tracers aerosolized in the conference room with a scaling to MS2 bacteriophage, which we have consolidated in terms of eACH.

Despite limitations such as the inability to aerosolize MS2 bacteriophage in the conference room setting, due to practicality, and the weak responsiveness of MS2 bacteriophage to 222 nm UVGI fixtures, the study demonstrated strong support for scaling and implementation in field testing. Furthermore, while DNA-tagged tracers currently model DNA damage only, they showed promising responsiveness to a broad range of UV-C wavelengths, including 222 nm wavelength exposure, suggesting potential for future testing with more susceptible viral surrogates sensitive to Far UV-C wavelengths and protein damage.

With in situ testing, the air mixing behavior of a room under study might not be well characterized. In situ environments might not be well mixed due to unique geometries, occupancy, furniture, variable locations of HVAC supplies and returns, and other unique situational factors. By aerosolizing the DNA-tagged tracers and measuring the response from a selected origin point for aerosol emission, along with an informed selection of the location of the air samplers, it is possible to get a representative view of what is happening at the location of the air samplers. In the case of the conference room, air samplers might be placed on a conference room table, a position that has a high chance of being occupied in such a scenario. The methods and apparatus described herein can provide a valuable snapshot of airflow and UVGI dynamics at select, highly pertinent points in the building, and the ability to scale those results with how MS2 bacteriophage, a viral surrogate, would be expected to respond to ventilation, filtration, and any UVGI installed in that space.

### 1. Foil Coupon Testing

Our investigation commenced utilizing foil coupons with dried DNA-tagged tracers to elucidate the responses of the tracers to UV exposure at different wavelengths of UV radiation. This format permitted a streamlined workflow to assess UV dose response of the tracers tested. These experiments not only describe the wavelength-dependence of the intrinsic kinetics of the photochemical reactions that are responsible for DNA detection decay, but also inform the interpretation of data from testing of the effects of UVGI fixtures in actual use settings. The results of these experiments illustrated LM4's heightened sensitivity to 282 nm radiation, relative to radiation at shorter wavelengths, a phenomenon potentially influenced by DNA absorption characteristics. Importantly, LM4 is capable of detecting UV response across all three wavelengths tested.

The DNA-tagged tracers in the tests were synthetic DNA and not encapsulated in a viral envelope or proteins. This is a limitation of the DNA tracer because UV damaging effects on proteins are not simulated by the DNA-tagged tracers, and only nucleic acid damage is simulated by the tracers. Nonetheless, the ability to accurately quantify the effects of UV exposure through a safe, inert aerosol that can be used in occupied spaces provides insights into commercial building applications, heretofore, not possible with other technologies like radiometric readings and inert gas release studies. Other tracers might be used that can provide for estimating protein damage to aerosol tracers.

As the data herein shows, with the UV-sensitive tracer, LM4, there is a point in the UV dose response behavior (at doses above 50 mJ/cm²), where there is a non-linearity in LM4 response not seen with the tracer D1. This nonlinearity appears as a “hockey stick” shape, with a leveling out of the decay response at these higher doses, as illustrated in FIG. 18. One possible explanation for this behavior is that the UV photodamage tracer LM4 has reached an equilibrium in the forward and reverse reaction of creation and undoing of photodamage, such as thymine dimer formation [30]. UV-induced photoproducts do not necessarily break the DNA backbone, and thus it is possible that at this energy regime, an equilibrium condition could develop. In this particular set of tests, only the linear portion of the dose response was considered in the subsequent analysis of the decay rates for LM4.

### 2. Aerosolized Testing in a 30 Cubic Meter Test Chamber

Transitioning to aerosolized testing in a 30 m³ controlled test chamber at 0 ACH conditions, the effectiveness of the UV-sensitive DNA-tagged tracer LM4 in responding to UV doses at different UVGI fixture wavelengths from different devices was assessed. The UV-resistant DNA-tagged tracer D1 exhibits a smaller and less accurate change of response for UV on/off compared to LM4, as expected for a more UV-resistant DNA tracer. This difference underscores the benefit of LM4 being sensitive to UV exposure. It might be expected that similar results would occur for different test chamber sizes, different ACH conditions, and different tracers.

Despite challenges in measuring the Krypton 222 nm UVGI device's reduction response with aerosolized bacteriophage MS2 in the test chamber, DNA-tagged tracer LM4 aerosolized in the same test chamber is able to detect the effect of 222 nm exposure, as shown in FIGS. 20 and 21. In this example of wavelength (222 nm) and DNA tracer Tag-LM4, it is interesting that the tracer outperforms the bacteriophage viral surrogate in sensitivity to UV doses in the Far UVGI fixture spectrum. Simultaneously releasing MS2 phage and aerosolized DNA-tracers Tag-D1 and Tag-LM4 in these tests, FIGS. 19-21 underscore Tag-LM4's superior sensitivity to the UVGI fixture at 222 nm, in comparison to the MS2 bacteriophage's response, serving as a human viral pathogenic surrogate, in the Far UVGI fixture spectrum (222 nm). These results imply that bacteriophage MS2 may not be a good surrogate for Far UV germicidal fixtures. Bacteriophage MS2 was chosen in this study for the reason that it best fit the guidelines of standards such as the recently published ASHRAE Standard 241-2023 [26]. As there was no large change in bacteriophage MS2 with the 222 nm device, the scaling factor is probably not entirely reliable for this regime of UVGI fixture. A more sensitive viral surrogate might be used for demonstrating the efficacy of Far UV technology with UV-sensitive DNA-tagged tracers. Ideal challenge agents might demonstrate UV dose-response behavior that is slightly conservative, as compared to target pathogens [31].

One observation of the test results is that the LED-based SUVOS-25 produced a powerful effect in the test chamber in terms of log reduction over time, as seen in FIG. 21 and Table 10 and Table 11 with tag LM-4, which was log-linear only within 25 minutes. After 25 minutes, the values did not follow the same reduction rate. There is a “hockey-stick” effect in the data after 25 minutes for SUVOS-25, similar to the “hockey-stick” non-linearity seen with Tag-LM4 at higher wavelengths and UV doses on foil coupon exposure as shown in FIG. 18. This effect could imply and contribute to inaccuracy when scaling between surrogate bacteriophage and DNA-tagged tracers of very high powered UVGI devices and might be taken into account. For example, the linear response range might only be scaled for a limited time period, such as the first 25 minutes, for Tag-LM4 with some device in some test chamber.

### 3. Comparison of Scaled MS2 Results in Test Chamber:

A scaling factor, derived by converting logarithmic reductions to equivalent air changes per hour, facilitated the assessment of UVGI fixture efficacy. The scaling factor, calculated on a per-fixture basis, represents the ratio of the delta in eACH between UV off and on conditions for both the bacteriophage and the tracer. This approach provides an understanding of the impact of UVGI fixtures on the DNA tracers and a facile way to scale to a pathogen surrogate, in this case MS2 bacteriophage.

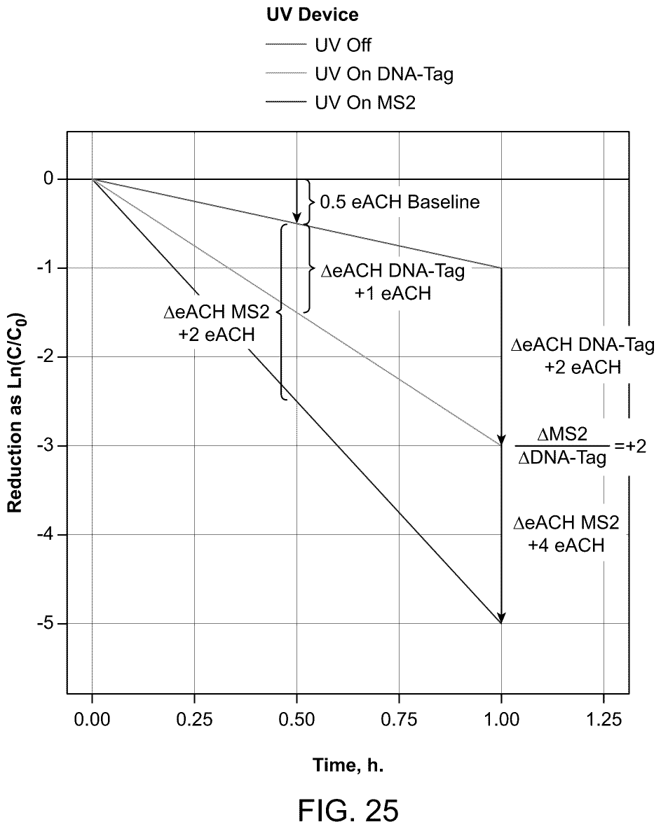

FIG. 25 illustrates scaling factors for MS2 and DNA-Tag. In FIG. 25, the top sloped line represents a baseline for the UV device being off. The middle sloped line represents the UV on DNA-Tag, while the lowest sloped line represents the UV On MS2. FIG. 25 provides a view on how the scaling factor between MS2 and DNA-Tag is kept constant if it is calculated as reduction rates on eACH unit, which is a logarithmic scale. This implies that, if the eACH for the DNA-Tag (whichever tag it might be) is known, the eACH for MS2 can be calculated. This might only be an accurate estimation if the decay rate of the DNA-Tag and MS2 are log-linear during the whole period of measurement, therefore, the data collected during experimentation should be inspected for this assumption.

Table 12 outlined the scaling of the responses to the four UVGI fixtures and their UV exposures in the controlled test chamber of the DNA-tagged tracers scaled to the same UV exposures of bacteriophage MS2. Tables 10-11 and FIGS. 20 and 21 illustrate the response of both DNA-tagged tracers to exposure to UV light from the four test fixtures tested in the controlled aerosol test chamber. For the DNA-tagged tracer Tag-D1, its resistance to UV-C decay significantly affects the accuracy of the scaling relationship, mathematically as the delta of the DNA-tagged tracer's response under “UV on” vs “UV off” conditions approach zero, the scaling factor goes to infinity, emphasizing the unsuitability of Tag-D1 as a surrogate due to its greater variability and lower sensitivity, compared with DNA-tagged tracer Tag-LM4. While comparing the scaled MS2 results for Tag-LM4 and Tag-D1 in Table 15, the proximity of their outcomes shows that Tag-D1 results are potentially useful, but in most of the cases, given the high scale relationship they could end in higher equivalent reduction rates. Thus, Tag-LM4 might provide a more accurate representation, in addition to its wider range of measurement of change given its sensitivity.

### 4. Commercial Building Conference Room Testing

Beyond controlled environments, these experiments in a commercial building conference room demonstrate adaptability of a DNA-tracer system. In the context of our findings, the differences between the AB24 device (a UVGI troffer fixture) in the commercial building application and controlled test chamber conditions were unexpected, with the AB24 device performing much better than the SUVOS-25 device in the commercial building setting. This highlights the importance of validating results in practical settings. Unique airflow geometries of the conference room, distinct from the continuous fans in the test chamber, likely contributed to these variations, as well as a troffer having its own fan at approximately 125 CFM (212.4 m³/h). Another possibility is that the SUVOS-25 device might be better scaled with Tag-D1, as it was linear over the full hour in the test chamber with Tag-D1 vs Tag-LM4 (see, for example, FIGS. 20-21). If this is the case, then the SUVOS-25 eACH is probably more accurately 6.22 vs. 4.68 for Tag-D1 and Tag-LM4 respectively, scaling with bacteriophage MS2 as the data in Table 15 shows. The test chamber experimentation provided an accurate effect of the tracers to UV exposure under controlled conditions, with exceptional mixing and 0 ACH by sealing the chamber. The conference room, by contrast, has its own unique geometry from the room design, HVAC supply and return positions, tables, chairs, and equipment. The conference room air sampler positions were chosen to be between the supply and return and situated on the conference room table, where people would most likely be seated at any given time while it is in use. In actual practice, the air samplers might be placed where people are likely to be and breathing the air, or other places in the environment that would provide representative sampling.

The same four UVGI fixtures used in the controlled chamber testing were applied to the conference room to assess the DNA-tagged tracers' responses to the fixtures' UV output over one hour. Table 15 shows a conversion of the conference room results scaled to MS2 bacteriophage in this scenario. The baseline eACH for the room, as determined by the no UV condition with Tag-D1 and Tag-LM4, was 2.27 eACH and 2.54 eACH respectively. All of the UVGI devices tested contributed to an increased eACH and when scaled to MS2 bacteriophage reduction, the total eACH of the room ranged from 2.83 to 7.87 eACH based on the MS2 scaling with Tag-LM4 tracer as shown by Table 15. These were significant improvements to the conference room's overall eACH, when translating to percentages, three devices Lexus, SUVOS-25, and AB24 increased the estimated MS2 decay rate to more than 99% reduction per hour (equivalent of 4.6 eACH total).

The air cleaning system equivalent clean airflow rate in CFM (VACS) was also calculated for Table 15, using Equation 3 in the methods. This calculates, according to ASHRAE Standard 241, the amount of equivalent clean airflow rate, at the air sampler positions, that air cleaning systems, in this case UVGI fixtures, are producing. The amount of equivalent clean air delivered to the air sampler locations on the conference room table ranged from 10.44 to 191.88 CFM after being scaled to bacteriophage MS2 (with Tag-LM4). In the ASHRAE 241 Standard, the recommended Equivalent Clean Air delivered to the space per person (ECAi) is 30 CFM for an office type space. Using this metric, the fixtures are adding enough CFM of equivalent clean air, at the conference room table, for the equivalent of an additional, approximate 0.4 to 6.4 people to be at the table.

Those results are after scaling the DNA tracer response to bacteriophage MS2. The Krypton 222 nm device is likely underrepresented in these results when scaling to bacteriophage MS2 in terms of its effectiveness (0.4 additional people at the table), since Far UV-C wavelengths might be scaled to more sensitive surrogate viruses. Nonetheless, the non-Far UV-C fixtures, when scaled to bacteriophage MS2, yield 2.6 (SUVOS-25), 4.6 (AB24), and 6.4 (Lexus 2.1) additional people to be at the conference room table based on the VAcs calculations for DNA Tag-LM4 scaled to bacteriophage MS2. This is a significant improvement to the room occupancy capability.

### 5. Ventilation Effectiveness, Guidelines and Standards

Aligning with recent CDC guidelines [32], advocating for a minimum of five air changes per hour (residing in the CDC Covid-19 guidelines), practical and quantitative method and apparatus for evaluating devices can provide equivalent ventilation effectiveness as described herein. Correlating log reductions of the DNA tracer and MS2 phage with a scaling factor, as detailed herein, can provide insights essential for establishing healthier indoor environments. Such a scaling could then be correlated to known log reductions of airborne pathogens to UV exposure from published literature, such as Kowalski [10]. This information can aid in determining whether a space meets or exceeds the recommended air changes per hour. Our test results can represent a level of risk protection in terms of equivalent air changes per hour (eACH) and alternatively, equivalent clean airflow rate in CFM (VACS). This is the estimation of the same pathogen decay rate that can be provided by different mechanisms such as ventilation and filtration, now parsed out in this case as germicidal UV-light contributions to healthier indoor environments [33].

As has been explained herein, methods and apparatus for measuring UV effectiveness and optimizing indoor air quality in diverse settings is provided. Test results from controlled experiments and commercial building scenarios highlight the versatility and adaptability of the DNA-based tracer system for evaluating germicidal systems described herein. These tests can be chemically inert and non-hazardous, which enables UV testing in occupied spaces. Precise measurements can be made on UV-sensitive DNA-tagged tracers and their response to UVGI fixtures by employing the highly quantitative droplet digital PCR methodology. By offering a nuanced understanding of the impact of UVGI fixtures on airborne particles and scaling the results to viral surrogates like bacteriophage MS2, this method allows a detailed understanding of UV contributions to public health and safety considerations in the built environment.

A correlation between log reductions of the DNA tracer and the MS2 bacteriophage, along with a scaling factor as described herein, can provide a quantitative measure of a room's unique ventilation and pathogen disinfection effectiveness. This aligns with recent CDC guidelines recommending a minimum of five air changes per hour to reduce the risk of exposure to pathogens, by providing a new technology that can be used to verify the performance of air cleaning interventions. The methods and apparatus described herein can provide a practical assessment of whether a space meets or exceeds the recommended air changes per hour, contributing to the establishment of healthier indoor environments.

### GENERALIZATION

In some embodiments, the data structures are used by various components and tools, some of which are described in more detail herein. The data structures and program code used to operate on the data structures may be provided and/or carried by a transitory computer readable medium, e.g., a transmission medium such as in the form of a signal transmitted over a network. For example, where a functional block is referenced, it might be implemented as program code stored in memory. The application can be one or more of the applications described herein, running on servers, clients or other platforms or devices and might represent memory of one of the clients and/or servers illustrated elsewhere.

According to some embodiments, the techniques described herein are implemented by one or more generalized computing systems programmed to perform the techniques pursuant to program instructions in firmware, memory, other storage, or a combination. Special-purpose computing devices may be used, such as desktop computer systems, portable computer systems, handheld devices, networking devices or any other device that incorporates hard-wired and/or program logic to implement the techniques.

One embodiment might include a carrier medium carrying data that includes data having been processed by the methods described herein. The carrier medium can comprise any medium suitable for carrying the data, including a storage medium, e.g., solid-state memory, an optical disk or a magnetic disk, or a transient medium, e.g., a signal carrying the data such as a signal transmitted over a network, a digital signal, a radio frequency signal, an acoustic signal, an optical signal or an electrical signal.

Various forms of media may be involved in carrying one or more sequences of one or more instructions to a processor for execution. For example, the instructions may initially be carried on a magnetic disk or solid-state drive of a remote computer. The remote computer can load the instructions into its dynamic memory and send the instructions over a network connection.

Operations of processes described herein can be performed in any suitable order unless otherwise indicated herein or otherwise clearly contradicted by context. Processes described herein (or variations and/or combinations thereof) may be performed under the control of one or more computer systems configured with executable instructions and may be implemented as code (e.g., executable instructions, one or more computer programs or one or more applications) executing collectively on one or more processors, by hardware or combinations thereof. The code may be stored on a computer-readable storage medium, for example, in the form of a computer program comprising a plurality of instructions executable by one or more processors. The computer-readable storage medium may be non-transitory. The code may also be provided carried by a transitory computer readable medium e.g., a transmission medium such as in the form of a signal transmitted over a network.

Conjunctive language, such as phrases of the form “at least one of A, B, and C,” or “at least one of A, B and C,” unless specifically stated otherwise or otherwise clearly contradicted by context, is otherwise understood with the context as used in general to present that an item, term, etc., may be either A or B or C, or any nonempty subset of the set of A and B and C. For instance, in the illustrative example of a set having three members, the conjunctive phrases “at least one of A, B, and C” and “at least one of A, B and C” refer to any of the following sets: {A}, {B}, {C}, {A, B}, {A, C}, {B, C}, {A, B, C}. Thus, such conjunctive language is not generally intended to imply that certain embodiments require at least one of A, at least one of B and at least one of C each to be present.

The use of examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

Further embodiments can be envisioned to one of ordinary skill in the art after reading this disclosure. In other embodiments, combinations or sub-combinations of the above-disclosed invention can be advantageously made. The example arrangements of components are shown for purposes of illustration and combinations, additions, re-arrangements, and the like are contemplated in alternative embodiments of the present invention. Thus, while the invention has been described with respect to exemplary embodiments, one skilled in the art will recognize that numerous modifications are possible.

For example, the processes described herein may be implemented using hardware components, software components, and/or any combination thereof. The specification and drawings are, accordingly, to be regarded in an illustrative rather than a restrictive sense. It will, however, be evident that various modifications and changes may be made thereunto without departing from the broader spirit and scope of the invention as set forth in the claims and that the invention is intended to cover all modifications and equivalents within the scope of the following claims.

What is claimed is:

1. A germicidal verification system comprising:
a dispersing unit for dispersing a tag composition, wherein the tag composition comprises two or more tags, a tag comprising a nonpathogenic portion having a particular range of sensitivity to exposure to ultraviolet (UV) radiation, wherein not all tags of the two or more tags have the same range of sensitivity;
a conduit for providing dispersed tags in the tag composition, for being dispersed into an environment;
a collection unit for collecting collected tag composition, wherein the collected tag composition comprises portions of the dispersed tags dispersed into the environment, exposed at least in part to some UV radiation in the environment, and then collected by the collection unit; and
a measurement unit for measuring amounts of the two or more tags in the collected tag composition, wherein measurements of the measurement unit of a given tag depend, at least in part, on an amount of sensitivity to UV exposure of the given tag.

2. The germicidal verification system of claim 1, wherein sensitivity to UV exposure of the given tag is due to UV radiation degradation of the given tag.

3. The germicidal verification system of claim 1, wherein the tags of the two or more tags comprise noncoding strings of nucleic acid base pairs.

4. The germicidal verification system of claim 3, wherein the noncoding strings of nucleic acid base pairs are selected from the group of: DNA sequences and RNA sequences.

5. The germicidal verification system of claim 1, wherein the conduit is adapted to disperse tags into or upon air, water, and/or a material that is to be sanitized.

6. The germicidal verification system of claim 1, wherein the measurements of the measurement unit measure concentrations of pathogen surrogates having differing UV sensitivities, whereby changes in pathogen surrogate concentrations due to UV exposure can be distinguished from changes in concentrations due to air mixing, dilution, flow obstructions, and/or introduction of non-tag materials.

7. The germicidal verification system of claim 1, wherein the tag comprises a primer portion, a tag identifying sequence, and a dose-responsive sequence.

8. The germicidal verification system of claim 7, wherein the tag composition comprises at least two tags that have different dose-responsive sequences.

9. The germicidal verification system of claim 8, wherein the different dose-responsive sequences are base pair sequences selected to control for sensitivity of a tag to UV degradation.

10. The germicidal verification system of claim 9, wherein the primer portion is selected to be relatively highly robust to UV exposure.

11. The germicidal verification system of claim 1, wherein tags are deployed in an aerosol form to be mixed with air that is to be sanitized.

12. The germicidal verification system of claim 1, wherein tags are deployed in a solid form and applied to coupons attachable to surfaces.

13. The germicidal verification system of claim 12, wherein the coupons are formed from a substrate having tags attached thereto, suitable for application to a surface to be sanitized using UV radiation exposure.

14. The germicidal verification system of claim 1, wherein the measurement unit for measuring amounts measures a first original concentration of a first tag, a second original concentration of a second tag, a first post-exposure concentration of the first tag, and a second post-exposure concentration of the second tag, and determines a UV germicidal effectiveness based on original versus measured concentrations of the first tag compared to original versus measured concentrations of the second tag, wherein the first tag and the second tag have differing robustness to UV exposure.

15. The germicidal verification system of claim 1, wherein tags are selected to have robustness to UV exposure that is determined based on an amount of expected UV exposure in the environment.
